SEQUENCE LISTING (30 sequences)

SEQ ID NO: 1 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccataccagaggaaatcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 2 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 3 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaagaacaacaccgtgaacgatctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccataccagaggaaatcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 4 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKNNTVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 5 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctaacaacaccctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccataccagaggaaatcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 6 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPNNTLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 7 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcaacttcaccgattacgaggaactgaaacatctgctgagtaggatcaatcactttgaaaaaattcagattatccccaaatcttcctggtcctcccatgaggcatctctgggcgtgtcatctgcctgtccataccagaggaaatcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 8 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGNFTDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 9 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcgacttcaacgattacgaggaactgaaacatctgctgagtaggatcaatcactttgaaaaaattcagattatccccaaatcttcctggtcctcccatgagaactcttctggcgtgtcatctgcctgtccataccagaggaaatcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 10 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHENSSGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 11 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccatacaacaggacctcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 12 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYNRTSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 13 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccataccagaggaactcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 14 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRNSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 15 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccataccagaggaaatcctcattcttccggaacgtggtgtggctcatcaaaaaaaactccacctaccccaccatcaaccgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 16 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTINRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 17 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccataccagaggaaatcctcattcttccggaacgtggtgtggctcatcaaaaaaaactccacctaccccaccatcaaacgctcttacaacaacacaaatcaggaggatctgctggtcctctggggaattcatcactctaatgatacagccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaatacgcctacaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 18 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHSNDTAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 19 - DNA, 1695 bp, Influenza virus
  CDS (1)..(1695)
atggagaaaattgtcctgctgttcgccattgtctcactggtcaaatccgatcagatctgtattggctaccacgccaacaatagcactgaacaggtcgacactattatggaaaaaaacgtgaccgtcacacatgctcaggatattctcgaaaaaacccacaacgggaaactctgtgatctcgacggagtgaaaccactcattctgagagactgtagcgtcgctggatggctcctcggcaatccaatgtgtgatgagttcatcaacgtccccgaatggtcatacatcgtggagaaggccaaccctgtgaacgatctctgttaccctggcgacttcaacgattacgaggaactgaaacatctgctgagtaggatcaatcactttgaaaaaattcagattatccccaaatcttcctggtcctcccatgaggcatctctgggcgtgtcatctgcctgtccataccagaggaaatcctcattcttccggaacgtggtgtggctcatcaaaaaaaactccacctaccccaccatcaaacgctcttacaacaacacaaatcaggaggatctgctggtcctctggggaattcatcaccccaatgatgccgccgagcagacaaaactgtaccagaaccctaccacatacatttctgtgggcacctctacactgaatcagaggctggtgcctagaattgccactaggagtaaagtcaacggccagtccggccggatggaattcttttggaccatcctcaaacccaacgatgctatcaacttcgagtcaaacggcaactttatcgcccctgaaaacgccaccaaaatcgtgaaaaagggcgactccactatcatgaaatccgagctggagtacggaaactgtaacaccaaatgccagacccctatgggcgctatcaactcttctatgcccttccacaacatccaccctctcactatcggcgaatgcccaaaatacgtcaaatcaaaccggctcgtgctggctactgggctgagaaactcacctcagcgagagactagaggcctgtttggcgccattgctggattcattgagggaggctggcagggaatggtcgatggctggtacggataccatcactccaatgagcagggatctggatacgctgccgataaggagtccacccagaaagcaatcgatggcgtcaccaacaaagtcaattcaatcatcgacaaaatgaacacccagttcgaggctgtgggacgagagttcaataacctggagcggagaatcgaaaacctgaacaaaaaaatggaggacggcttcctcgatgtgtggacctacaatgctgaactgctggtgctcatggaaaacgagagaaccctggacttccacgactcaaacgtgaaaaacctgtacgacaaagtccggctccagctgagggataatgccaaggaactcggaaatggctgcttcgagttctaccacaaatgtgacaacgagtgtatggagtctgtccgaaacggaacctacgactaccctcagtactctgaggaggctagactgaaacgagaggagatctctggcgtcaaactggagtctatcggaatctaccagattctgtccatctactctactgtggcttcttcactggctctggccatcatggtcgctgggctgtctctgtggatgtgctcaaatggatcactccagtgccggatctgtatctag

SEQ ID NO: 20 - protein, 564 aa, Influenza virus
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQRKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPENATKIVKKGDSTIMKSELEYGNCNTKCQTPMGAINSSMPFHNIHPLTIGECPKYVKSNRLVLATGLRNSPQRETRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 21 - DNA, 759 bp, Influenza virus
  CDS (1)..(759)
atgagtcttctaaccgaggtcgaaacgtatgttctctctatcgtcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacaacaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccattgggactcatcctagctccagtgctggtctaaaagatgatcttcttgaaaatttgcaggcctatcagaaacgaatgggggtgcagatgcaacgattcaagtga

SEQ ID NO: 22 - protein, 252 aa, Influenza virus
MSLLTEVETYVLSIVPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVTTTNPLIRHENRMVLASTTAKAMEQMAGSSEQAAEAMDIASQARQMVQAMRTIGTHPSSSAGLKDDLLENLQAYQKRMGVQMQRFK

SEQ ID NO: 23 - DNA, 294 bp, Influenza virus
  CDS (1)..(294)
atgagtcttctaaccgaggtcgaaacgcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaa

SEQ ID NO: 24 - protein, 97 aa, Influenza virus
MSLLTEVETPIRNEWGCRCNDSSDPLVIAANIIGILHLILWILDRLFFKCIYRRFKYGLKRGPSTEGVPESMREEYRKEQQNAVDVDDGHFVNIELE

SEQ ID NO: 25 - DNA, 1860 bp, Artificial
  cDNA of FliC-M2 fusion protein
atgaaattcttagtcaacgttgcccttgtttttatggtcgtgtacatttcttacatctatgcggccgcacaagtcattaatacaaacagcctgtcgctgttgacccagaataacctgaacaaatcccagtccgctctgggcaccgctatcgagcgtctgtcttccggtctgcgtatcaacagcgcgaaagacgatgcggcaggtcaggcgattgctaaccgttttaccgcgaacatcaaaggtctgactcaggcttcccgtaacgctaacgacggtatctccattgcgcagaccactgaaggcgcgctgaacgaaatcaacaacaacctgcagcgtgtgcgtgaactggcggttcagtctgctaacagcaccaactcccagtctgacctcgactccatccaggctgaaatcacccagcgcctgaacgaaatcgaccgtgtatccggccagactcagttcaacggcgtgaaagtcctggcgcaggacaacaccctgaccatccaggttggtgccaacgacggtgaaactatcgatatcgatctgaagcagatcaactctcagaccctgggtctggatacgctgaatgtgcaacaaaaatataaggtcagcgatacggctgcaactgttacaggatatgccgatactacgattgctttagacaatagtacttttaaagcctcggctactggtcttggtggtactgaccagaaaattgatggcgatttaaaatttgatgatacgactggaaaatattacgccaaagttaccgttacggggggaactggtaaagatggctattatgaagtttccgttgataagacgaacggtgaggtgactcttgctggcggtgcgacttccccgcttacaggtggactacctgcgacagcaactgaggatgtgaaaaatgtacaagttgcaaatgctgatttgacagaggctaaagccgcattgacagcagcaggtgttaccggcacagcatctgttgttaagatgtcttatactgataataacggtaaaactattgatggtggtttagcagttaaggtaggcgatgattactattctgcaactcaaaataaagatggttccataagtattaatactacgaaatacactgcagatgacggtacatccaaaactgcactaaacaaactgggtggcgcagacggcaaaaccgaagttgtttctattggtggtaaaacttacgctgcaagtaaagccgaaggtcacaactttaaagcacagcctgatctggcggaagcggctgctacaaccaccgaaaacccgctgcagaaaattgatgctgctttggcacaggttgacacgttacgttctgacctgggtgcggtacagaaccgtttcaactccgctattaccaacctgggcaacaccgtaaacaacctgacttctgcccgtagccgtatcgaagattccgactacgcgaccgaagtttccaacatgtctcgcgcgcagattctgcagcaggccggtacctccgttctggcgcaggcgaaccaggttccgcaaaacgtcctctctttactgcgtggaggaggaggaggaggaatgagtcttctaaccgaggtcgaaacgcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaa

SEQ ID NO: 26 - protein, 619 aa, Artificial
  Synthetic Construct
MKFLVNVALVFMVVYISYIYAAAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEDVKNVQVANADLTEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLRGGGGGGMSLLTEVETPIRNEWGCRCNDSSDPLVIAANIIGILHLILWILDRLFFKCIYRRFKYGLKRGPSTEGVPESMREEYRKEQQNAVDVDDGHFVNIELE

SEQ ID NO: 27 - DNA, 1350 bp, Influenza virus
  CDS (1)..(1350)
atgaatccaaatcagaagataataaccatcggatcaatctgtatggtaactggaatagttagcttaatgttacaaattgggaacatgatctcaatatgggtcagtcattcaattcacacagggaatcaacaccaatctgaaccaatcagcaatactaattttcttactgagaaagctgtggcttcagtaaaattagcgggcaattcatctctttgccccattaacggatgggctgtatacagtaaggacaacagtataaggatcggttccaagggggatgtgtttgttataagagagccgttcatctcatgctcccacttggaatgcagaactttctttttgactcagggagccttgctgaatgacaagcactccaatgggactgtcaaagacagaagccctcacagaacattaatgagttgtcctgtgggtgaggctccctccccatataactcaaggtttgagtctgttgcttggtcagcaagtgcttgccatgatggcaccagttggttgacgattggaatttctggcccagacaatggggctgtggctgtattgaaatacaatggcataataacagacactatcaagagttggaggaacaacatactgagaactcaagagtctgaatgtgcatgtgtaaatggctcttgctttactgtaatgactgacggaccaagtaatggtcaggcatcacataagatcttcaaaatggaaaaagggaaagtggttaaatcagtcgaattggatgctcctaattatcactatgaggaatgctcctgttatcctaatgccggagaaatcacatgtgtgtgcagggataattggcatggctcaaatcggccatgggtatctttcaatcaaaatttggagtatcaaataggatatatatgcagtggagttttcggagacaatccacgccccaatgatggaacaggtagttgtggtccggtgtcctctaacggggcatatggggtaaaagggttttcatttaaatacggcaatggtgtctggatcgggagaaccaaaagcactaattccaggagcggctttgaaatgatttgggatccaaatgggtggactgaaacggacagtagcttttcagtgaaacaagatatcgtagcaataactgattggtcaggatatagcgggagttttgtccagcatccagaactgacaggactagattgcataagaccttgtttctgggttgagttgatcagagggcggcccaaagagagcacaatttggactagtgggagcagcatatctttttgtggtgtaaatagtgacactgtgggttggtcttggccagacggtgctgagttgccattcaccattgacaagtag

SEQ ID NO: 28 - protein, 449 aa, Influenza virus
MNPNQKIITIGSICMVTGIVSLMLQIGNMISIWVSHSIHTGNQHQSEPISNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDK

SEQ ID NO: 29 - DNA, 798 bp, Artificial
  cDNA of  PRO-M2 fusion protein
tccgactgggaccctgttgtcaaggagtggcttgttgacacaggctactgctgcgcaggcggcatcgccaacgcggaggacggtgttgtgttcgccgcggcggctgatgatgatgacggatggtccaagctgtacaaggatgatcatgaggaggacactatcggagaggatggcaacgcgtgcggcaaggtgtcgatcaacgaggcctccacgatcaaagctgcagttgacgatggcagtgcccctaacggtgtttggattggcggccagaagtacaaggttgtccgacctgagaaaggattcgagtacaacgactgcaccttcgacatcaccatgtgtgcacggtccaagggtggcgcgcacttgatcaagaccccgaatggctctatcgtcattgccctttacgatgaggagaaggaacaggacaagggaaacagcaggacttcggcattggcctttgccgagtatcttcaccagtctgggtacggaggaggaggaggaggaatgagtcttctaaccgaggtcgaaacgcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaa

SEQ ID NO: 30 - DNA, 729 bp, Artificial
  cDNA of GM-CSF-M2 fusion protein
tggctccagaacctgctgtttctcggcattgtcgtgtactcactgtccgccccaactcgatctcctatcaccgtcactagaccttggaaacacgtggaggcaatcaaagaggccctgaatctgctcgacgatatgcctgtgacactgaacgaggaagtggaagtggtgtccaatgagttctccttcaaaaaactcacctgtgtccagactagactgaaaatcttcgaacagggactccgggggaatttcaccaaactgaaaggcgctctgaacatgactgcttcatactaccagacctactgcccacccacacctgaaaccgattgtgagactcaggtcaccacatacgccgacttcatcgatagcctcaaaacattcctcaccgacatcccatttgagtgtaaaaaacccggccagaaaggaggaggaggaggaatgagtcttctaaccgaggtcgaaacgcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaa

(19) United States
(12) Patent Application Publication
(10) Pub. No.: US 2014/0221628 A1
(43) Pub. Date: Aug. 7, 2014

(54) INFLUENZA VIRUS-LIKE PARTICLES COMPRISING ADJUVANT-FUSED M2 PROTEIN TO ENHANCE THE IMMUNOGENICITY OF VACCINE

(71) Applicant: National Tsing Hua University, Hsinchu (TW)

(72) Inventors: Suh-Chin Wu, Hsinchu (TW); Wen-Chun Liu, Hsinchu (TW); Hung-Ju Wei, Hsinchu (TW)

(73) Assignee: NATIONAL TSING HUA UNIVERSITY, Hsinchu (TW)

(21) Appl. No.: 14/247,254

(22) Filed: Apr. 8, 2014

Related U.S. Application Data
(63) Continuation-in-part of application No. 13/449,654, filed on Apr. 18, 2012.

(51) Int. Cl. C07K 14/005

(52) U.S. Cl. CPC C07K 14/005
USPC 530/403

(57) ABSTRACT

A DNA vaccine comprising hyperglycosylated mutant HA gene, which is derived from avian influenza virus, is provided. A DNA vaccine composition comprising: (a) the DNA vaccine; and (b) a booster is also provided. An influenza virus-like particle comprising adjuvant-fused M2 protein is further provided. A method for eliciting an immune response against a plurality of avian influenza virus subtypes in a subject, comprising delivering the DNA vaccine or the DNA vaccine composition to tissue of the subject is also provided

### CROSS-REFERENCES TO RELATED APPLICATIONS

The present application is a continuation-in-part application which claims priority to U.S. application Ser. No. 13/449,654, filed Apr. 18, 2012, incorporated herein by reference in its entirety.

### FIELD OF THE INVENTION

The present invention relates to a DNA vaccine. More specifically, the present invention relates to a DNA vaccine comprising hyperglycosylated antigen.

The present invention also relates to a DNA vaccine composition and a method for eliciting an immune response against multiple avian influenza virus subtypes in a subject using the same. The present invention further relates to an influenza virus-like particle comprising adjuvant-fused M2 protein, also a method for eliciting stronger immune response against multiple avian influenza virus subtypes in a subject.

The sequence listing text file, file name 2116_NTHU_SQ, created Apr. 7, 2014, file size 172021 bytes, is incorporated herein by reference in its entirety.

### BACKGROUND OF THE INVENTION

Highly pathogenic avian influenza (HPAI) H5N1 viruses and their capacity for transmission from birds to humans have raised worldwide concerns about a potential forthcoming human pandemic. With the continued spread of H5N1 influenza virus, new virus strains have emerged and will continue to change and evolve in the future. The World Health Organization has classified the H5N1 viruses isolated recently into 10 clades (or sublineages) based on the phylogenetic analysis of viral hemagglutinin (HA) sequences of H5N1 viruses. With the continuous threat of a new influenza pandemic arising from avian reservoirs, the development of broadly protective vaccines is particularly important. To date, the broadly protective H5N1 vaccines have been mainly achieved using novel adjuvant formulations.

However, the inherent nature of influenza virus antigenic changes has not been taken into accounts in the immunogen designs for developing broadly protective H5N1 vaccines. Refocusing antibody responses have been proposed by designing the immunogens that can preserve the overall fold of the immunogen structure but selectively mutate the “undesired” antigenic sites that are highly variable (escape mutants evade protective immune responses), immunosuppressive (downregulate the immune response to the infection), cross-reactive (the immune response induces a reaction to a protein resembling the immunogen). The immunogen design by refocusing antibody responses has been applied for HIV-1 vaccines using the hyperglycosylated HIV-1 gp120 immunogens where the undesired eptiopes are masked by selective incorporations of N-linked glycans. The glycan masking strategy has been also recently reported to design influenza virus vaccines that can enhance the antibody responses against a broad range of H3N2 intertypic viruses. However, there is no report for the use of glycan-masking immunogen design for H5N1 vaccines.

DNA vaccine has been considered as the revolutionary vaccinology with the advantages in offering genetically antigen design, time to manufacturing, long stability without the need for cold chains supply, and the immunogenicity predominantly elicited by T cells through the endogenerous antigen processing pathways. However, the apparent low immunogenicity of DNA vaccines in large animals (including humans) has been overcome using novel delivery systems such as gene-guns or electroporation. Additionally, the DNA vaccine-elicited immune responses can be further augmented using the heterologous prime-boost immunization regimen where the booster dose uses a different vaccine format containing the same or similar antigens. Examples of DNA vaccine prime-boost immunization strategy has been reported for the inactivated influenza virus, live-attenuated influenza virus, recombinant adenovirus, virus-like particles (VLPs) and recombinant subunit proteins in adjuvants. Furthermore, human vaccines receiving the H5 DNA vaccine priming followed by a booster with inactivated H5N1 vaccine were found to enhance the protective antibody responses (HAI) and in some cases induce the haemagglutinin-stem-specific neutralizing antibodies.

Influenza VLPs are noninfectious and have a size and morphology that are similar to those of native virion structures, but they do not contain the genomic RNAs for virus replication. The assembly of influenza VLPs depends on the interactions of M1 proteins and/or other viral surface proteins, such as HA, NA, and M2, with the cellular lipid membranes. The interactions of M1 protein with the cytoplasmic tails of HA and NA spikes can increase the lipid membrane binding of M1 proteins in assembling influenza virus. The interactions of HA and NA with the M1 protein can also reduce the formation of elongated intracellular immature particles and improve the secretion of spherical mature VLPs. Additionally, the cytoplasmic tails of M2 protein, by interacting with the M1 protein, further promote the budding and release of the influenza virions. Recently, the M2 protein was found to act as the plasma membrane-targeting signal for the budding and egress of influenza virions. Host cell proteins can be recruited into the VLPs, as recently shown by LC/MS/MS analyses. Therefore, the biosynthesis of influenza VLPs is a self-assembly process that involves complex interactions of viral and cellular components. Additionally, the VLPs containing adjuvant(s)-fused M2 proteins vaccine-elicited immune responses can be further augmented using the prime-boost immunization regimen.

### BRIEF DESCRIPTION OF THE DRAWINGS

The invention can be more fully understood by reading the subsequent detailed descriptions and examples with references made to the accompanying drawing, wherein:

FIG. 1 shows expression and characterization of DNA-HA and FliC-VLP. (A) The cell lysates of 293A cells transfected with either DNA-HA or empty vector were treated with Endo H, PNGase F and Trypsin, and analyzed by Western blots. Full-length HA proteins showed the presence of a molecular weight of approximately 75 kDa and HA1 proteins showed the presence of a molecular weight of about 46 kDa. (B) FliC-VLPs were purified by sucrose gradient sedimentation and the results showed the fractions 6 to 10 from the sucrose density gradient contained all four proteins. (C) Electron microscopic visualization demonstrated the spherical morphology of the FliC-VLPs with a particle size around 100 nm.

FIG. 2 shows total anti-HA IgG titers elicited by DNA-HA and FliC-VLP. Asterisks indicate a statistically significant difference (p<0.05).

FIG. 3 shows neutralizing activities of the sera from immunized mice by the (A) HI and (B) NT titers against the NIBRG-14 (clade 1) H5N1 influenza virus. For calculation purposes, an undetectable level was scored as a titer equal to one. Individual titer (points) and geomean (lines) was given for each group.

FIG. 4 shows analytical result of amino acid variation in the HA of 163 avian influenza virus strains. Eleven amino acids in the HAI subunit, including the 83, 86, 94, 124, 129, 138, 140, 155, 162, 189 and 252 residues were calculated to have relatively higher scoring numbers.

FIG. 5 shows nine N-linked glycosylation sites: 83NNT (SEQ ID NO:4), 86NNT (SEQ ID NO:6), 94NFT (SEQ ID NO:8), 127NSS (SEQ ID NO:10), 138NRT (SEQ ID NO:12), 140NSS (SEQ ID NO:14), 161NRS (SEQ ID NO:16), 182NDT (SEQ ID NO:18), and 252NAT (SEQ ID NO:20). Underlined triplet amino acids and arrows point away from wild-type sequence to amino acid change that resulted in N-linked glycosylation sequence.

FIG. 6 shows the results of hemadsorption assay. (A) Positive control; (B) negative control; (C) 83NNT; (D) 86NNT; (E) 94NFT; (F) 127NSS; (G) 138NRT; (H) 161NRS; (I) 182NDT; and (J) 252NAT.

FIG. 7 shows characterization of hyperglycosylated HA. The six HA mutant proteins (83NNT, 86NNT, 94NFT, 127NSS, 138NRT, 161NRS) with N-linked glycans addition were illustrated by the increased molecular weights and reduced to the same molecular weight after PNGase F treatment.

FIG. 8 shows total anti-HA IgG titers elicited by hyperglycosylated HA. Individual titer (points) and geomean (lines) was given for each group.

FIG. 9 shows neutralizing activities of sera from immunized mice by the (A) HI and (B) NT titers against the NIBRG-14 (clade 1) H5N1 influenza virus. For calculation purposes, an undetectable level was scored as a titer equal to one. Individual titer (points) and geomean (lines) was given for each group. Asterisks indicate a statistically significant difference (p<0.05).

FIG. 10 shows neutralizing activities of sera from immunized mice by the (A) HI and (B) NT titers against the Mongolia/2/2006 (clade 2.2) H5N1 influenza virus. For calculation purposes, an undetectable level was scored as a titer equal to one. Individual titer (points) and geomean (lines) was given for each group. Asterisks indicate a statistically significant difference (p<0.05).

FIG. 11 shows construction of baculovirus expression vector for influenza VLP production. Influenza VLPs are obtained from Sf9 cells that are infected with (A) a single baculovirus that encodes two viral proteins (BacHA-M1) (B) two baculoviruses that encode three viral proteins (BacHA-M1 and BacNA) (C) two baculoviruses that encode four viral proteins (BacHA-M1 and BacNA-M2). pH: polyhedron promoter; p10: p10 promoter.

FIG. 12 shows sucrose gradient analyses of the influenza VLPs obtained by the expression by baculovirus of (A) two viral proteins (HA and M1); (B) three viral proteins (HA, NA, M1); and (C) four viral proteins (HA, NA, M1, M2). Purified sucrose fractions were resolved in SDS-PAGE gels and reacted with anti-HA, anti-M1, anti-NA, and anti-M2 antibodies.

FIG. 13 shows TEM analyses of influenza VLPs expressed by baculovirus using (A-D) two viral proteins (HA and M1); (E-H) three viral proteins (HA, NA, M1); and (I-L) four viral proteins (HA, NA, M1, M2). The TEM images present quadruple samples for each case of negative staining of influenza VLPs with uranyl acetate.

FIG. 14 shows production of influenza VLPs with EGFP/M2 fusion protein. (A) Sucrose gradient analysis of influenza VLPs, reacted with anti-HA, anti-NA, anti-M1, anti-EGFP specific antibodies; (B-E) TEM images of influenza EGFP-VLPs that are negatively stained with uranyl acetate, showing quadruple samples.

FIG. 15 shows EGFP-VLPs in A549 cells visualized by confocal fluorescence microscopy. A549 cells were labeled with DiD and EGFP-VLPs were labeled with DiI. (A) Excitation by 488 nm line from laser and 633 nm line from laser; (B) excitation by 561 nm line from laser and 633 nm line from laser.

FIG. 16 shows production of influenza VLPs with FliC/M2 fusion protein. (A) Sucrose gradient analysis of influenza VLPs reacted with anti-HA, anti-NA, anti-M1, anti-M2 specific antibodies; (B-E) TEM images of influenza FliC-VLPs that are negatively stained with uranyl acetate, showing quadruple samples.

FIG. 17 shows production of influenza VLPs with PRO/M2 fusion protein. (A) Sucrose gradient analysis of the influenza VLPs reacted with anti-HA, anti-NA, anti-M1, and anti-M2 specific antibodies; and (B) TEM images of influenza PRO-VLPs that are negatively stained with uranyl acetate, showing quadruple samples.

FIG. 18 shows intracellular TNF-α production of BMDCs treated with (A) non-fabricated VLPs, (B) FliC-VLPs, (C) PRO-VLPs, (D) PBS (negative control), or (E) 20 ng/mL LPS (positive control). TNF-α production was detected by FACS analysis in groups of treated (black lines) and untreated (gray lines) BMDCs. Average TNF-α+ BMDCs of gated M1 were obtained from at least three independent experiments.

FIG. 19 shows analytic results of CD40 and CD86 surface markers on BMDCs treated with non-fabricated VLPs, FliC-VLPs and PRO-VLPs. The mean fluorescence intensity (MFI) of the groups of treated (black lines) and untreated (gray lines) BMDCs are presented in (A) CD40⁺CD11c⁺ and (B) CD86⁺CD11c⁺ phenotypes. Results are obtained from triplicate experiments.

FIG. 20 shows neutralization of antisera collected from mice immunized with VLPs, FliC-VLPs and PRO-VLPs using H5 pp of (A) the homologous KAN-1 strain and (B) the heterologous Anhui strain.

FIG. 21 shows the sucrose gradient analyses of wild-type and adjuvanted-fused influenza VLPs obtained by insect cells-baculovirus expression system. (A) WT VLPs containing four viral strucure proteins HA, NA, M1 and M2, (B) VLPs containing M2 fusion protein of GM-CSF, (C) VLPs containing M2 fusion protein of FliC, and (D) VLPs containing M2 fusion protein of GM-CSF/FliC. Purified sucrose fractions were resolved in SDS-PAGE gels and reacted with anti-HA, anti-M1, anti-NA, and anti-M2 antibodies.

FIG. 22 shows antibody responses elicited by WT VLPs and adjuvanted VLPs (GMCSF VLPs, FliC VLPs and GMCSF/FliC VLPs) in immunized female mice. (A) is the immunization regimen, (B) is a chart showing anti-HA IgG and IgM titer and IgG subclasses (IgG1 and IgG2a) titer, and (C) is a chart showing IgG1/IgG2a ratio. Data were represented as mean±standard error. Results were analyzed using student's T tests, asterisks (**) indicate the statistical significance p<0.01.

FIG. 23 is a chart showing influenza virus-specific antibodies secreting B cells induced by WT VLPs and adjuvanted-VLPs (GMCSF VLPs, FliC VLPs or GMCSF/FliC VLPs) in the spleens of immunized female mice. Data were represented as mean±standard deviation. The asterisk indicates the statistical significance p<0.05.

FIG. 24 shows HI antibodies titers induced by WT VLPs and adjuvanted-VLPs (GMCSF VLPs, FliC VLPs or GMCSF/FliC VLPs) immunization against (A) the homologous KAN-1 strain and (B) the heterologous Qinghai strain of the H5 pseudotyped particles (H5 pp). Data were represented as mean±standard error. Results were analyzed using student's T tests, the asterisk indicates p<0.05.

FIG. 25 shows neutralization titers of sera collected from mice immunized with WT VLPs and adjuvanted-VLPs (GMCSF VLPs, FliC VLPs or GMCSF/FliC VLPs) against (A) the homologous KAN-1 strain, (B) the heterologous Qinghai strain, and (C) the heterologous Anhui strain of the H5 pseudotyped particles (H5 pp). (D), (E) and (F) show LogIC50 values of neutralization curves. ** indicate p<0.01; *** indicate p<0.001.

FIG. 26 shows the ELISPOT analyses of T cell responses elicited by WT VLPs and adjuvanted-VLPs (GMCSF VLPs, FliC VLPs or GMCSF/FliC VLPs). (A) shows IFN-γ T cell response. (B) shows IL-4 T cell response. Data were expressed as the mean±SEM of five animals per group. * indicates p<0.05; ** indicate p<0.01.

### SUMMARY OF THE INVENTION

The present invention relates to a DNA vaccine comprising hyperglycosylated HA gene(s), which is derived from avian influenza virus, wherein the mutant HA gene encodes a protein having a mutation at amino acid residue selecting from the group consisting of 83, 86, 94, 127, 138, 161, 182, and 252. The present invention also relates to a DNA vaccine composition comprising: (a) an above-mentioned DNA vaccine; and (b) a booster. The present invention further relates to an influenza VLP comprising adjuvant-fused M2 protein.

### DETAILED DESCRIPTION OF THE INVENTION

As used herein, the term “wild-type” refers to a naturally occurring organism. The term also relates to nucleic acids and proteins found in a naturally occurring organism of a naturally occurring population arising from natural processes, such as seen in polymorphisms arising from natural mutation and maintained by genetic drift, natural selection and so on, and does not include a nucleic acid or protein with a sequence obtained by, for example, recombinant means.

“Immunogen” and “antigen” are used interchangeably herein as a molecule that elicits a specific immune response of antibody (humoral-mediated) and/or T cell origin (cell-mediated), for example, containing an antibody that binds to that molecule or a CD4⁺ or CD8⁺ T cell that recognizes a virally-infected cell expressing that molecule. That molecule can contain one or more sites to which a specific antibody or T cell binds. As known in the art, such sites are known as epitopes or determinants. An antigen can be polypeptide, polynucleotide, polysaccharide, a lipid and so on, as well as a combination thereof, such as a glycoprotein or a lipoprotein. An immunogenic compound or product, or an antigenic compound or product is one which elicits a specific immune response, which can be humoral, cellular or both.

An “individual” or “subject” or “animal”, as used herein, refers to vertebrates that support a negative strand RNA virus infection, specifically influenza virus infection, including, but not limited to, birds (such as water fowl and chickens) and members of the mammalian species, such as canine, feline, lupine, mustela, rodent (racine, and murine, etc.), equine, bovine, ovine, caprine, porcine species, and primates, the latter including humans.

As used herein, the term “a plurality of” is employed to describe the number of elements and components of the present invention. This description should be read to more than one unless it is obvious that it is meant otherwise.

As used herein, the term “a” or “an” is employed to describe elements and components of the invention. This is done merely for convenience and to give a general sense of the invention. This description should be read to include one or at least one and the singular also includes the plural unless it is obvious that it is meant otherwise.

As used herein, the term “or” is employed to describe “and/or”.

Accordingly, the present invention provides a DNA vaccine comprising hyperglycosylated HA gene(s), which is derived from avian influenza virus, wherein the mutant HA gene encodes a protein having one or more mutations at amino acid residue selecting from the group consisting of 83, 86, 94, 127, 138, 161, 182, 252, and the combination thereof.

In one embodiment, the hyperglycosylated HA gene encodes a protein comprising an amino acid sequence selected from the group consisting of SEQ ID NOs: 4, 6, 8, 10, 12, 14, 16, 18, and 20. In another embodiment, the mutant HA gene encodes a protein comprising an amino acid sequence of SEQ ID NOs: 4, 6, or 10.

In one embodiment, delivery of the DNA vaccine into a subject elicits an immune response against a plurality of avian influenza virus subtypes in the subject. In another embodiment, the delivery is achieved by way of, for example but not limited to, subcutaneous injection, intramuscular injection, oral administration, spraying or gene gun injection.

The present invention also provides a DNA vaccine composition comprising: (a) an above-mentioned DNA vaccine; and (b) a booster.

In one embodiment, the booster is influenza VLP. In another embodiment, the influenza VLP is derived from cell infected by recombinant baculoviruses comprise one or more plasmids containing HA gene, M1 gene, NA gene and FliC-M2 gene, which encodes FliC-M2 fusion protein.

In one embodiment, the DNA vaccine composition further comprises an adjuvant. In another embodiment, the adjuvant is an aluminum-containing adjuvant.

In one embodiment, the DNA vaccine and the booster have a mass ratio in the range of 1:2 to 17:6. In another embodiment, the DNA vaccine and the booster have a mass ratio in the range of 5:6 to 5:2. In still another embodiment, the DNA vaccine and the booster have a mass ratio of 5 to 3.

In one embodiment, delivery of the DNA vaccine composition into a subject elicits an immune response against a plurality of avian influenza virus subtypes in the subject. In another embodiment, the delivery is achieved by way of, for example but not limited to, subcutaneous injection, intramuscular injection, oral administration, spraying or gene gun injection.

The present invention further provides an influenza VLP comprising adjuvant-fused M2 protein. In one embodiment, the influenza VLP further comprises HA protein, NA protein and M1 protein. In another embodiment, the adjuvant is flagellin (FliC) or profiling (PRO).

Besides aforementioned TLR ligands, FliC and PRO, other immunostimulatory cytokines have also been reported to enhance the immune responses of DNA vaccines against HIV/SIV and influenze viruses (Skountzou I, Quan F S, Gangadhara S, Ye L, Vzorov A, Selvaraj P, Jacob J, Compans R W, Kang S M. Incorporation of glycosylphosphatidylinositol-anchored granulocyte-macrophage colony-stimulating factor or CD40 ligand enhances immunogenicity of chimeric simian immunodeficiency virus-like particles. Journal of Virology, February 2007, p. 1083-1094; Loudon P T, Yager E J, Lynch D T, Narendran A, Stagnar C, Franchini A M, Fuller J T, White P A, Nyuandi J, Wiley C A, Murphey-Corb M, Fuller D H. GM-CSF increases mucosal and systemic immunogenicity of an H1N1 influenza DNA vaccine administered into the epidermis of non-human primates. PLoS One. 2010 Jun. 8; 5(6):e11021; Yao Q, Fischer K P, Li L, Agrawal B, Berhane Y, Tyrrell D L, Gutfreund K S, Pasick J. Immunogenicity and protective efficacy of a DNA vaccine encoding a chimeric protein of avian influenza hemagglutinin subtype H5 fused to CD 154 (CD40L) in Pekin ducks. Vaccine. 2010 Nov. 29; 28(51):8147-56; Rongxin Zhang, Sheng Zhang, Min Li, Changyi Chen, and Qizhi Yao. Incorporation of CD40 Ligand into SHIV Virus-Like Particles (VLP) Enhances SHIV-VLP-Induced Dendritic Cell Activation and Boosts Immune Responses against HIV. Vaccine. 2010 July 12; 28(31): 5114-5127; Lai L, Kwa S, Kozlowski P A, Montefiori D C, Ferrari G, Johnson W E, Hirsch V, Villinger F, Chemareddi L, Earl P L, Moss B, Amara R R, Robinson H L. Prevention of infection by a granulocyte-macrophage colony-stimulating factor co-expressing DNA/modified vaccinia Ankara simian immunodeficiency virus vaccine. The Journal of Infectious Diseases 2011; 204:164-73). Accordingly, in one embodiment of the present invention, the influenza virus-like particle is derived from cell infected by recombinant baculoviruses comprising one or more plasmids containing a gene encoding an adjuvant-fused M2 protein, wherein the adjuvant is flagellin (FliC), profilin (PRO), granulocyte macrophage colony-stimulating factor (GM-CSF), or a combination thereof. For example, the gene can be FliC-M2 gene (such as SEQ ID NO: 25), PRO-M2 gene (such as SEQ ID NO: 29), or GM-CSF-M2 gene (such as SEQ ID NO: 30), wherein FliC-M2 gene encodes FliC-M2 fusion protein, PRO-M2 gene encodes PRO-M2 protein, and GM-CSF-M2 gene encodes GM-CSF-M2 fusion protein. In another embodiment of the present invention, the adjuvant of the influenza VLP comprising adjuvant-fused M2 protein is flagellin (FliC), profilin (PRO), granulocyte macrophage colony-stimulating factor (GM-CSF), or a combination thereof. Still in another embodiment, the adjuvant is a combination of GM-CSF and FliC.

In one embodiment, delivery of the adjuant(s)-fused VLP vaccine into a subject elicits an immune response against a plurality of avian influenza virus subtypes in the subject. In another embodiment, the delivery is achieved by way of, for example but not limited to, subcutaneous injection, intramuscular injection, oral administration, spraying or gene gun injection.

The next examples provide some exemplary embodiments of the present invention as follows:

### EXAMPLES

The examples below are non-limiting and are merely representative of various aspects and features of the present invention.

### Example 1

### Material and Methods

### Construction of DNA-HA Vaccine Vector

The cDNA of the HA gene of influenza virus A/Thailand/1(KAN-1)/2004/H5N1 (clade 1), SEQ ID NO: 1, was provided by Prasert Auewarakul, Siriraj Hospital, Thailand. The full-length HA sequence was inserted into a pcDNA™ 3.1(+) vector (Invitrogen) using KpnI/NotI cut site. The constructed plasmid containing H5HA was transfected into 293A cells by using Turbofect reagent (Fermentas). Following transfection for 48 hours, the cell lysates were collected by centrifugation at 5000 rpm for 10 minutes and HA expression was analyzed by Western blotting with anti-H5HA antibodies (ab21297; Abcam).

### HA Glycosylation Pattern and Trypsin Treatment

For characterizing the HA glycosylation pattern, 293A cells were harvested after transfected with DNA-HA vectors for 48 hours. The cell lysates were treated with EndoH or PNGase F for 2 hours at 37° C., and the H5HA glycosylation pattern was determined by Western blotting. For trypsin treatment, the cell lysates were incubated with trypsin for 30 minutes on ice, and the cleavage of HA0 into HA1 and HA2 was observed by Western blotting.

### Preparation of VLPs

VLPs were prepared as described previously (Wei H J et al., Vaccine 29 (2011): 7163-7172). Briefly, HA (SEQ ID NO: 1) and M1 (SEQ ID NO: 21) were cloned into a pFastBac™ Dual vector (Invitrogen), while NA (SEQ ID NO: 27) and FliC-M2 (SEQ ID NO: 25), expressing FliC-M2 fusion proteins, were cloned into the other one to produce the recombinant baculoviruses. Sf9 cells co-infected with recombinant baculoviruses were harvested at 72 hours post-infection, and supernatants containing FliC-VLPs were concentrated by filtration with a 500 kDa filter membrane. The concentrate were loaded on 0-60% sucrose gradients and centrifuged for 4 hours at 33,000 rpm. The desired particles were observed by Western blotting using anti-H5HA antibodies (ab21297; Abcam), anti-NA antibodies (ab70759; Abcam), anti-M1 antibodies (ab25918; Abcam), and anti-M2 antibodies (NB100-2073; Novus). The particles were also confirmed by transmission electron microscopy (TEM) as described previously (Wei H J et al., Vaccine 29 (2011): 7163-7172).

### Preparation of Hyperglycosylated H5HA

Mutations were introduced into the HA gene by using the site-directed mutagenesis, and plasmids encoding wild-type H5HA gene (SEQ ID NO: 1) were used as templates. The 50 μL PCR reaction was carried out with 100 ng templates, 2 mM primer pair, 200 mM dNTPs and 2 U of DNA polymerase. The PCR products were purified and further treated with Dpnl for 2 hours at 37° C. Dpnl treated products were transformed into TOP10 competent cell and then the mutated plasmids were isolated.

### Hemadsorption Assay

293A cells were transfected with wild-type and mutated H5HA DNA vectors, and the cells were harvested at 72 hours post infection. Following phosphate-buffered saline (PBS) wash, sufficient 0.5% turkey red blood cells (RBCs) were added to cover cell monolayer and incubate for 30 minutes. Adsorption of RBCs on the transfected cells was observed after rinse with PBS two times.

### Mouse Immunization

6 to 8 weeks old female BALB/c mice were immunized with heterologous prime-boost strategy by 50 μg of DNA and 30 μg of purified VLPs mixed with Alum adjuvant in PBS. Immunizations were performed at weeks 0, 3 by intramuscular injection. Blood was collected at 14 days following immunization, and serum was isolated. Serum samples were inactivated at 56° C. for 30 minutes and stored in −20° C. All experiments were conducted in accordance with the guidelines of the Laboratory Animal Center of National Tsing Hua University (NTHU). Animal use protocols were reviewed and approved by the NTHU Institutional Animal Care and Use Committee (approval no. 09733).

### Enzyme-Linked Immunosorbent (ELISA) Assay

ELISA assay was performed as described previously (Lin S C et al., PLoS One 6 (2011): e20052). Briefly, 2 μg/mL of purified protein were coated on 96 well plates and then blocked with BSA. Serial dilutions of each serum sample were incubated in the plates for 1 hour and removed by 3 times wash. Goat anti-mouse IgG conjugated HRP (Bethyl Laboratories, Inc.) was incubated in the plates for 1 hour followed by 3 times wash. After the reaction with TMB substrate stop, plates were read at 450 nm absorbance. End-point titer was determined as the reciprocal of the final dilution giving an optical of two-fold absorbance of negative control.

### Hemagglutinin Inhibition (HI) and Neutralization (NT) Assays

HI and NT assays were performed as described previously (Huang M H et al., PLoS One 5 (2010): e12279). For HI assay, serum samples (two-fold dilutions starting with an initial dilution of 1:10) were incubated with four HA units of influenza strain. Turkey RBCs were then added and the inhibition of agglutination was scored. The serum titer was expressed as the reciprocal of the highest dilution that showed complete inhibition of HA. For NT assay, the 200 TCID₅₀ per well of virus were incubated with two-fold-diluted mice sera at a starting dilution of 1:40. Mixtures of virus and serum were transferred to monolayers of MDCK cells and incubated for 4 days. The neutralizing titer was defined as the reciprocal of the highest serum dilution at which the infectivity of the H5N1 virus was neutralized in 50% of the wells. Infectivity was identified by the presence of cytopathy on Day 4 and the titer was calculated using the Reed-Muench method.

### Statistic Analysis

All results were analyzed using two-tailed Student's t tests, with a P value of <0.05 indicating statistical significance

### Results

### Construction and Characterization of DNA-HA Vaccine Vector and FliC-VLPs for Prime-Boost Immunization

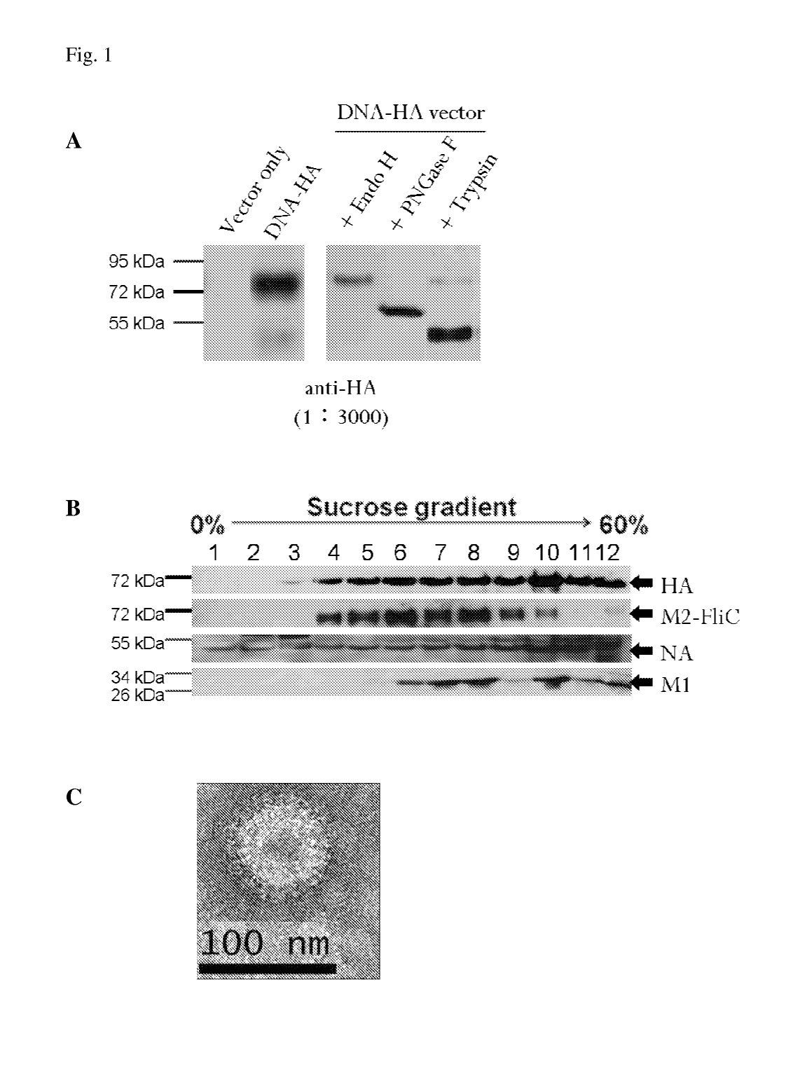

The DNA vaccine vector (DNA-HA) encoding the full-length cDNA of the A/Thailand/1 (KAN-1)/2004/H5N1 (clade 1) HA gene (SEQ ID NO: 1) was constructed from the pcDNA™3.1(+) vector. Expression of the full-length HA protein was demonstrated in 293A cells transfected with the DNA-HA vector and analyzed in Western blots to show the presence of a molecular weight of approximately 75 kDa (FIG. 1A). The expressed HA in 293A cells was sensitive to PNGase F treatment but resistant to EndoH digestion, suggesting as a glycoprotein containing complex type N-linked glycan profiling (FIG. 1A). The expressed HA in DNA-HA transfected 293A cells was also sensitive to trypsin treatment by cleavage from HA0 to HA1 and HA2 subunits, as shown the presence of HA1 at a molecular weight about 46 kDa (FIG. 1A).

The FliC-containing VLPs (FliC-VLPs) were obtained from Sf9 cells infected with two recombinant baculoviruses encoding four of the influenza virus genes of HA, NA, and M1, and the fusion of M2 and the Samollena fliC genes (Wei H J et al., Vaccine 29 (2011): 7163-7172). FliC-VLPs were obtained from the culture supernatants of baculovirus-infected Sf9 cells, purified by ultracentrifugation and sucrose gradient sedimentation. The results show the fractions 6 to 10 from the sucrose density gradient contained all four viral or fusion proteins (FIG. 1B). Electron microscopic visualization demonstrated the spherical morphology of the FliC-VLPs with a particle size around 100 nm (FIG. 1C).

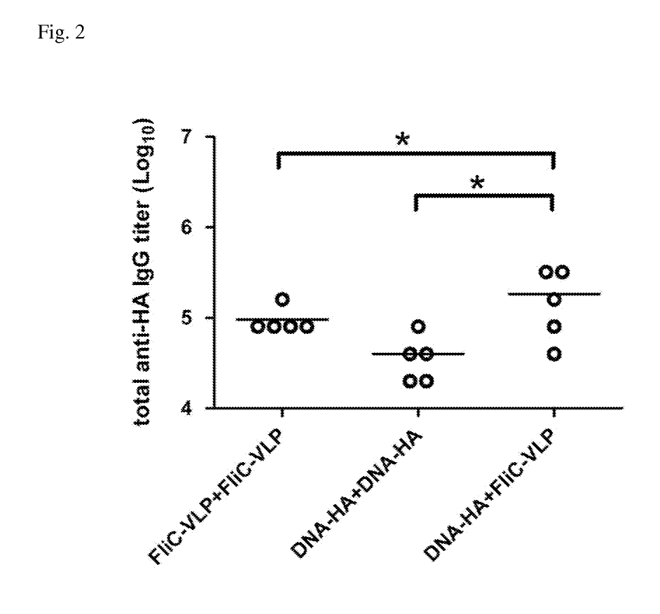

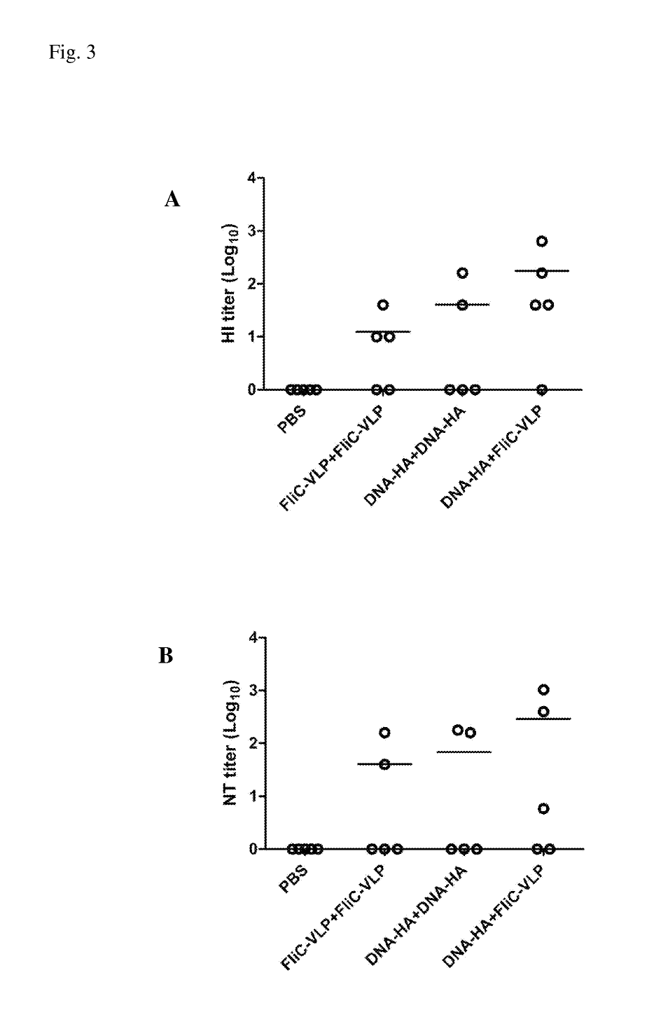

To investigate the combined use of DNA-HA vaccine vector and FliC-VLP for prime-boost immunization studies, BALB/c mice were immunized intramuscularly (i.m) for two doses within a three-week interval as the following prime-boost regimens: (i) PBS+PBS (ii) FliC-VLP+FliC-VLP (iii) DNA-HA+DNA-HA (iv) DNA-HA+FliC-VLP. Sera were collected at two weeks after the second dose in immunized mice. The results show that the HA-specific total IgG titer by DNA-HA vaccine vector priming, followed by FliC-VLP boosting was significantly higher than two-dose immunization using DNA-HA vector and FliC-VLPs (FIG. 2). Neutralizing activities revealed by measuring the HI and NT titers against the NIBRG-14 (clade 1) H5N1 influenza virus show that the DNA-HA vector priming and FliC-VLP boosting regiment elicited the highest magnitude of neutralizing antibodies in mice (FIGS. 3A-B).

### Design of Hyperglycosylated HA Based on Amino Acid Sequences of H5N1 Human Isolates

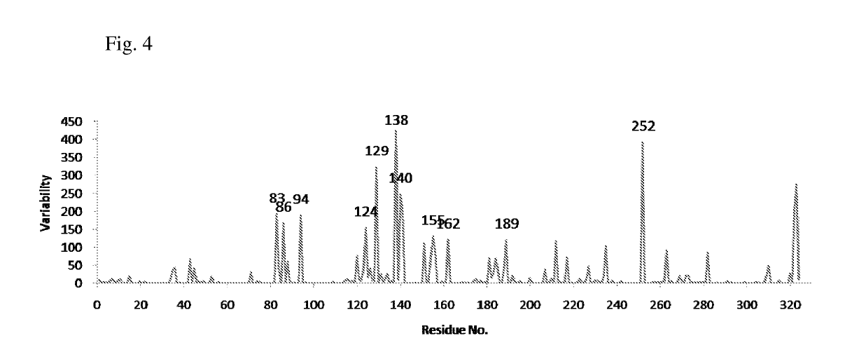

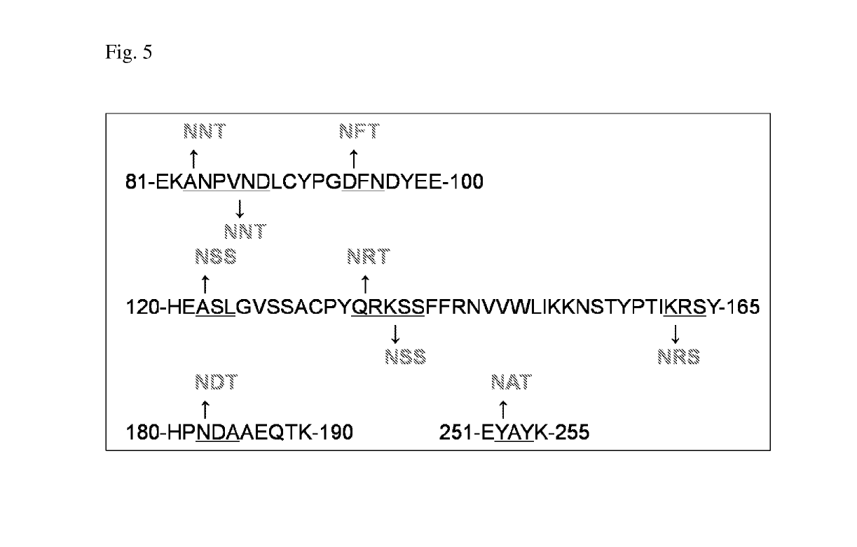

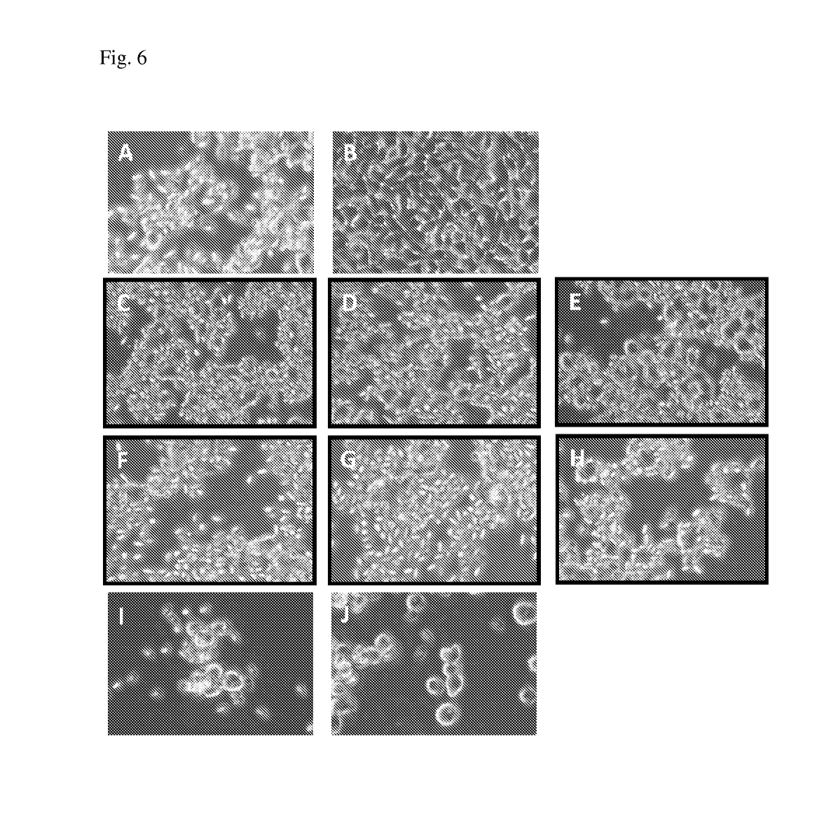

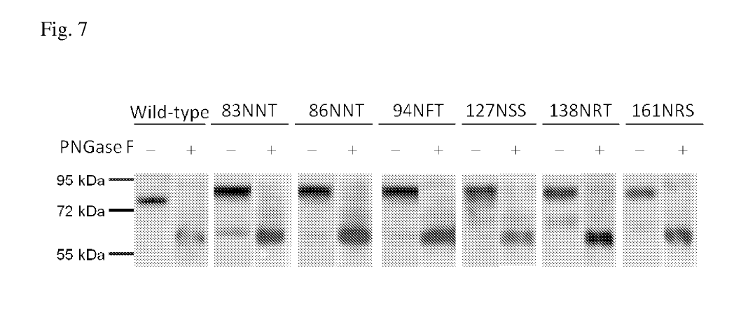

To design the hyperglycosyalted HA DNA vaccines, sequence alignment analysis was first conducted from 163 HPAI H5N1 human isolates (sequences retrieved from NCBI Database). The amino acid differences in these HA1 protein sequences were analyzed based on the following scoring numbers, 4 (different amino acid), 2 (weak similar amino acid), 1 (strong similar amino acid), 0 (identical amino acid) as characterized by the Vector NTI Similar Tables. According to the alignment plot shown in FIG. 4, eleven amino acid residues in the HAI protein were identified to have a relatively higher scoring numbers, including the 83, 86, 94, 124, 129, 138, 140, 155, 162, 189, and 252 residue. To design the antibody-refocused immunogens, site-directed mutagenesis is conducted in each of the five regions with mutations to allow the addition of the N-X-S/T motif (for N-linked glycosylation site) but avoid the receptor binding sites (Yang Z Y et al., Fig. Science 317 (2007): 825-828; and Yang H et al., PLoS Pathog 6 (2010): e1001081). Nine N-X-S/T motifs were thus introduced into HA1, including 83NNT (SEQ ID NO: 4), 86NNT (SEQ ID NO: 6), 94NFT (SEQ ID NO: 8), 127NSS (SEQ ID NO: 10), 138NRT (SEQ ID NO: 12), 140NSS (SEQ ID NO:14), 161NRS (SEQ ID NO: 16), 182NDT (SEQ ID NO: 18), and 252 NAT (SEQ ID NO: 20) (FIG. 5). Each of the refocusing hyperglycosylated HA genes containing the specified N-linked glycosylation sites were cloned into the DNA-HA vaccine vector. However, only six out the nine immunofocusing HA retained the hemagglutination property for Turkey red blood cells after transfection into 293A cells (FIG. 6). The six HA mutant genes (83NNT, 86NNT, 94NFT, 127NSS, 138NRT and 161NRS) were also investigated for the introduction of N-linked glycans in the HA antigens as illustrated by the increased molecular weights and reduced to the same molecular weight after PNGase F treatment (FIG. 7).

Priming with hyperglycosylated HA DNA vaccines followed by FliC-VLP boosting

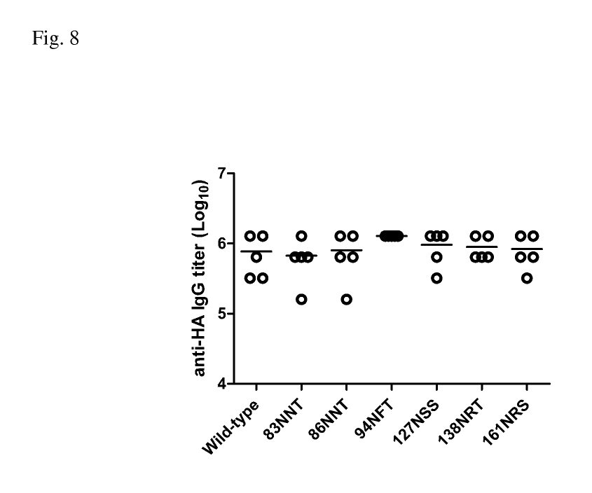

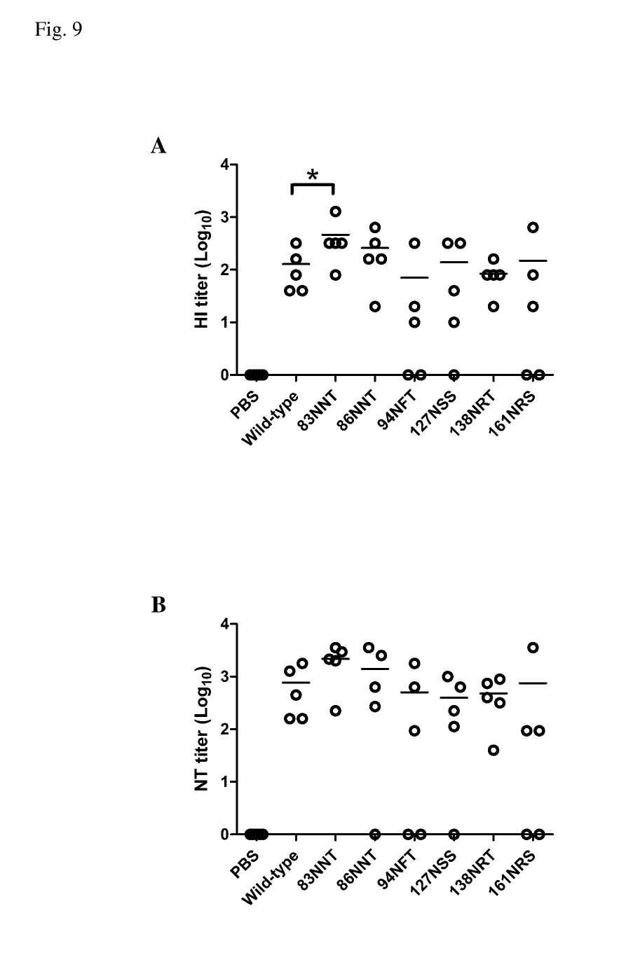

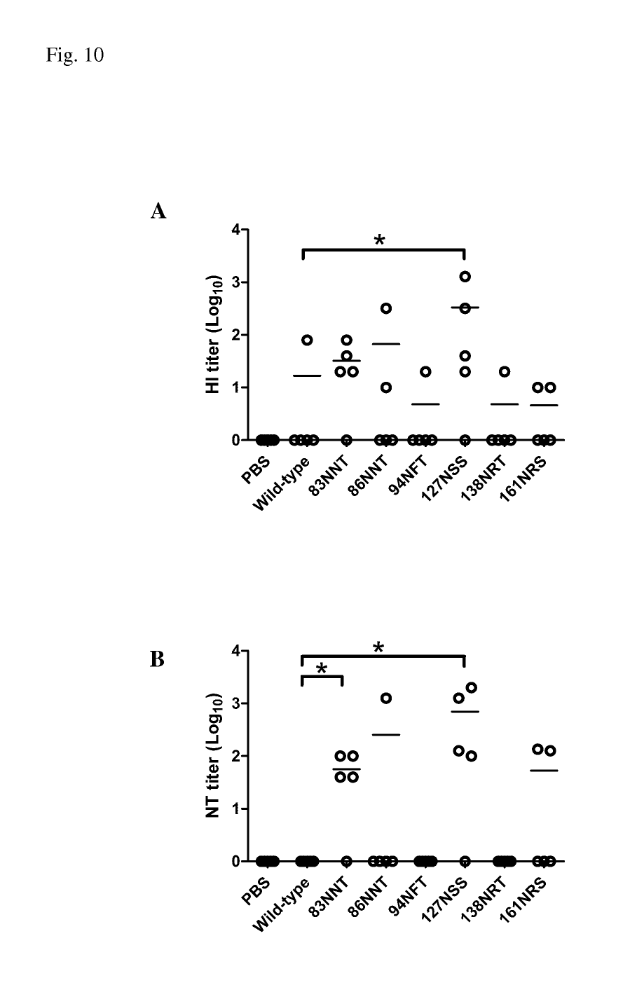

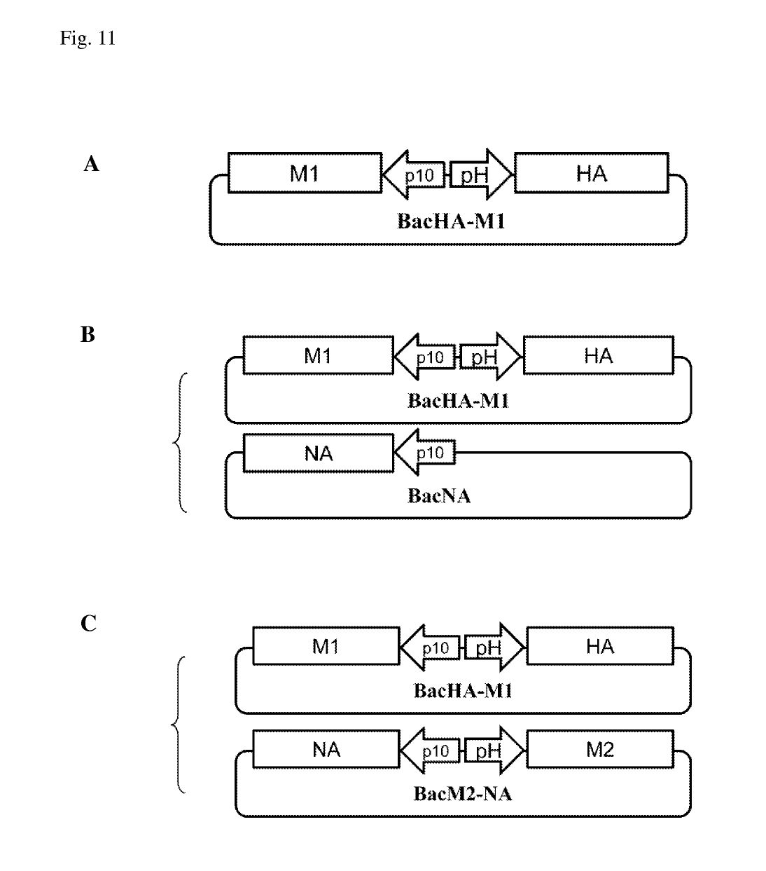

To investigate the antibody responses elicited by these six hyperglycosylated HA mutants (83NNT, 86NNT, 94NFT, 127NSS, 138NRT and 161NRS), mice were immunized with each DNA-HA vector twice followed with a third boosting dose with FliC-VLPs on a three-week interval. The results show that no significant differences of the HA-specific total IgG titers of all the immunized groups with the hyperglycosyalted HA DNA vaccines compared to the wild-type control (FIG. 8). The 83NNT and 86NNT HA mutants elicited higher HI titers (FIG. 9A) but only the 83NNT HA mutant had higher NT titer (FIG. 9B) against the NIBRG-14 virus that belongs to the same H5N1 clade 1 strain. The HI and NT titers of these sera against the Mongolia/2/2006 H5N1 virus of the clade 2.2 strain were also measured. The data presenting as cross-clade functional antibodies show that the 83NNT, 86NNT, 127NSS HA mutants elicited higher HI titers (FIG. 10A) and the 83NNT, 86NNT, 127NSS, 161 NRS HA mutants had higher NT titers (FIG. 10B). Taken together, the 83NNT mutant can elicit more potent HI and NT titers against both the NIBRG-14 (clade 1) and Mongolia/2/2006 (clade 2.2) HPAI H5N1 viruses.

### Example 2

### Methods and Materials

### Cell Lines

Sf9 cells (ATCC CRL-1711) (Invitrogen) were derived from pupal ovarian tissue of the fall armyworm, Spodoptera frugiperda. Sf9 cells were maintained in T-flasks at 28° C. with SF-900II serum free medium (GIBCO) that contained 100 units/mL penicillin and 100 μg/mL streptomycin (Invitrogen). For suspension cultures, Sf9 cells were inoculated in 500 mL spinner flasks (Belleco) at 60 rpm at 27° C. with 300 mL of the same medium. A549 cells (human lung carcinoma cells) (ATCC CCL-185) were maintained in T-flasks at 37° C. with DMEM (GIBCO) that contained 5% fetal bovine serum (FBS), 100 units/mL penicillin, and 100 μg/mL streptomycin (Invitrogen).

### Mouse Bone Marrow-Derived DCs

C57BL/6 mice were used at 10-14 weeks of age and their bone marrow cells were isolated from femurs and tibias and seeded on Costar 24-well cell culture plates in 1 mL of RPMI 1640 medium that was also supplemented with 10% heat-inactivated FBS, 2 mM 1-glutamine, nonessential amino acids, sodium pyruvate, HEPES (all from GIBCO), 5.5×10⁻²M 2-ME (Sigma-Aldrich), 100 units/mL penicillin, 100 μg/mL streptomycin (Invitrogen) and 15 ng/mL recombinant mouse GM-CSF (PeproTech). On Day 3, 1 mL of medium that contained 10 ng/mL of GM-CSF was added to plates. On Day 5, another 0.5 mL fresh medium that contained 10 ng/mL of GM-CSF was added. The 6- to 7-day-culture BMDCs (>80% CD11c+ cells) were used. All experiments were conducted in accordance with the guidelines of Laboratory Animal Center of National Tsing Hua University (NTHU). The animal use protocols have been reviewed and approved by the NTHU Institutional Animal Care and Use Committee (Approved protocol no. 09733).

### Plasmid Construction

The HA gene of A/Thailand/1(KAN-1)/2004/H5N1 (SEQ ID NO: 1) was provided by Dr. Prasert Auewarakul, Siriraj Hospital, Mahidol University, Thailand. The NA gene of A/Viet Nam/1203/2004/H5N1 (SEQ ID NO: 27) was obtained from Academia Sinica, Taiwan. The M1 (SEQ ID NO: 21) and M2 (SEQ ID NO: 23) genes of A/WSN/33/H₁N₁ were obtained from virus stocks using reverse transcription-PCR. The genes of HA (A/Anhui/1/2005/H5N1), enhanced florescence protein (EGFP), flagellin (FliC), and profilin (PRO) were purchased from synthesized sequences (Mr. Gene) based on the NCBI GenBank accession numbers GU983383.1, AY649721.1 and AY937257.1, respectively. Each gene fragment was subcloned into pFastbac Dual (Invitrogen) using BamHI/NotI site for HA, XhoI/KpnI site for M1, EcoRI/HindIII site for M2, XhoI/KpnI site for NA, EcoRI/HindIII site for EGFP/M2 fusion, EcoRI/HindIII site for FliC/M2 fusion, and EcoRI/HindIII site for PRO/M2 fusion. These inserted vectors were then transformed into E. coli strain DH5α and selected by ampicillin. All the inserted sequences were confirmed by DNA sequence analysis (Mission Biotech Inc., Taipei, Taiwan).

### Generation of Recombinant Baculoviruses

The pFastbac Dual plasmids encoding each specified gene(s) were transformed into E. coli strain DH10Bac (Invitrogen) and selected on an LB plate that contained kanamycin (Invitrogen), gentamicin (Invitrogen), tetracycline (Invitrogen), Bluo-gal (Invitrogen), and IPTG (BioRad). The selected colonies or the recombinant bacmids were confirmed by PCR using M13 primers, then transfected into Sf9 cells using Cellfectin (Invitrogen). After 4 days, the recombinant baculoviruses were collected from culture supernatants and the virus titers were determined using an ID50 software.

### Production and Purification of Influenza VLPs

The VLPs that were expressed by two viral proteins and Sf9 cells were infected with BacHA-M1 recombinant baculovirus at an MOI of 1. The VLPs that were expressed by three viral proteins were co-infected with BacHA-M1 and Bac-NA recombinant baculoviruses at an MOI of 3 and 1, respectively. The VLPs that were expressed by four viral proteins including M2 fusion proteins were co-infected with BacHA-M1 and BacM2-NA (or BacEGFP/M2-NA, BacNA-M2/FliC, BacNA-M2/PRO) recombinant baculoviruses at an MOI of 3 and 1, respectively. At 72 hours post infection, the culture supernatants were harvested and clarified by centrifugation for 0.5 hour at 12,000 rpm at 4° C. Then, they were concentrated and pelleted for 2 hours at 33,000 rpm and 4° C. using a Hitachi RPS40ST rotor. The particles were resuspended in 0.8 mL of PBS buffer, and loaded on a 0-60% (w/v) discontinuous sucrose gradient, before being ultracentrifuged by a Hitachi RPS40ST rotor 4 hours at 33,000 rpm and 4° C. Following ultracentrifugation, the fractions (0.8 mL) were collected and the samples in each fraction were analyzed by SDS-PAGE and Western blotting.

### Hemagglutination Titer

For the hemagglutination titer test, a series of two-fold dilutions of influenza VLPs in PBS were prepared and incubated at 25° C. for 40 min with 50 μL of 0.5% Turkey red blood cells. The extent of hemagglutination was observed visually, and the highest dilution that can agglutinate red blood cells was determined.

### Sodium Dodecyl Sulfate Polyacrylamide Gel Electrophoresis (SDS-PAGE) and Western Blotting

Each sucrose gradient fraction sample was treated with 1×SDS gel-loading buffer (50 mM Tris-HCl, 100 mM dithiothreitol, 2% SDS, 0.1% bromophenol blue, and 10% glycerol) for 5 min, resolved on 12% SDS-PAGE, and then transferred to PVDF membranes. Following the transfer, the PVDF membranes were blocked using 10% milk on an orbital shaker for 1 hour. Then the membranes were first reacted with anti-HA (Abcam ab21297), anti-M1 (Abcam ab25918), anti-NA (Abcam ab70759), anti-M2 (novus NB100-2073) or anti-EGFP (novus NB-600-601ss) antibodies for 1 hour, then reacted with the goat anti-rabbit or goat anti-mouse IgG conjugated with HRP (horse radish peroxidase) for 1 hour. Enhanced chemiluminescence (ECL) was detected through binding to HRP and visualized on a Fuji Medical X-ray film using a Western blot detection system (Amersham Bioscience).

### Transmission Electron Microscopy (TEM)

The purified sucrose fractions containing VLPs were pooled and ultracentrifugated using the Hitachi RPS40ST rotor 2 hours at 33,000 rpm and 4° C. to remove the sucrose and to pellet the VLPs. The VLP pellets were resuspended with 200 μL PBS. For deep staining of the grid, 3 μL purified VLPs was added to the carbon-coated copper grid and stained three times with uranyl acetate before being vacuum-dried overnight.

### Confocal Fluorescence Microscopy

A549 cells were grown on glass coverslips. VLPs were labeled with DiI (Vybrant DiI cell labeling solution) and A549 cells were labeled with DiD (Vybrant DiD cell labeling solution). Labeled VLPs were incubated with labeled A549 cells and analyzed by confocal fluorescence microscopy. DiI was excited by the 561 nm line of a laser. DiD was excited by the 633 nm line of a laser. EGFP was excited by the 488 nm line of a laser.

### Mouse Immunization

A group of five female BALB/c mice (6 to 8 weeks old) was used for immunization studies. Immunizations were performed by intramuscular injection of 15 μg of the purified VLPs (suspended in PBS at pH 7.4) for each dose and three doses were conduced in a 3-week interval. Blood was collected 2 weeks after third immunization and serum was isolated. All experiments were conducted in accordance with the guidelines of the Laboratory Animal Center of National Tsing Hua University (NTHU). Animal use protocols were reviewed and approved by the NTHU Institutional Animal Care and Use Committee (approval no. 09733).

### H5-Pseudotyped Particles (H5 pp)

3×10⁶ HEK293T cells were transfected with pNL-Luc-E⁻R⁻, pcDNA3.1-HA (A/Thailand/1 (KAN-1)/2004/H5N1 and A/Anhui/1/2005/H5N1) and pcDNA4B-NA (A/Viet Nam/1203/2004/H5N1) vectors. Cell supernatant that contained pseudotyped HIV-1 particles with H5N1 HA and NA were collected 48 hours post-transfection and purified through a 0.45 μm filter. The supernatant was concentrated by ultracentrifugation at 33,000 rpm for 2.5 hours, and then each pellet was dissolved in 100 μL PBS. An HIV-1 p24 ELISA assay kit (BioChain) was used to quantify the H5 pp particles.

### Neutralization Assay

MDCK cells (4000 cells/well) were seeded in 100 μL of DMEM in 96-well plates. The amount of 25 ng of p24 H5 pp was incubated with two-fold serial dilutions of serum (starting dilution 1:40) for 1 hour at 37° C. in 60 μL DMEM. Then 100 μL of fresh medium was added and 140 μL of the virus-serum mixtures was transferred to the cells. The luciferase assay was performed 48 hours following the direct addition of neolite luciferase substrate (PerkinElmer). The neutralization titer was defined as the reciprocal of the dilution that yielded 50% neutralization determined using an ID50 software.

### Analysis of Cytokine Production

DCs were untreated or individually treated with LPS 50 ng/mL from E. coli 0111:B4 (Sigma), PBS, 1 μg/mL VLP, FliC-VLP or PRO-VLP for 6 hours, with the addition of a protein transport inhibitor, brefeldin A (10 μg/mL) (Biolegend), for the final 4.5 hours. Cells were then fixed and permeabilized, and the intracellular cytokines were stained with TNF-α mAb (Biolegend). They then underwent flow cytometry (FACS Calibur, BD) and analyzed using CellQuest software (BD Biosciences).

### Analysis of DC Maturation

After the BMDCs were untreated or treated with VLPs, FliC-VLPs or PRO-VLPs (5 μg/mL) for 16 hours, the cells and supernatants were harvested and stained with monoclonal antibodies against conjugated CD11c-FITC, conjugated CD40-PE, and conjugated CD86-PE (Biolegend). The cells were then acquired and analyzed using flow cytometry (FACS Calibur, BD).

### Results

### Baculovirus-Insect Cell Expression of Influenza VLPs

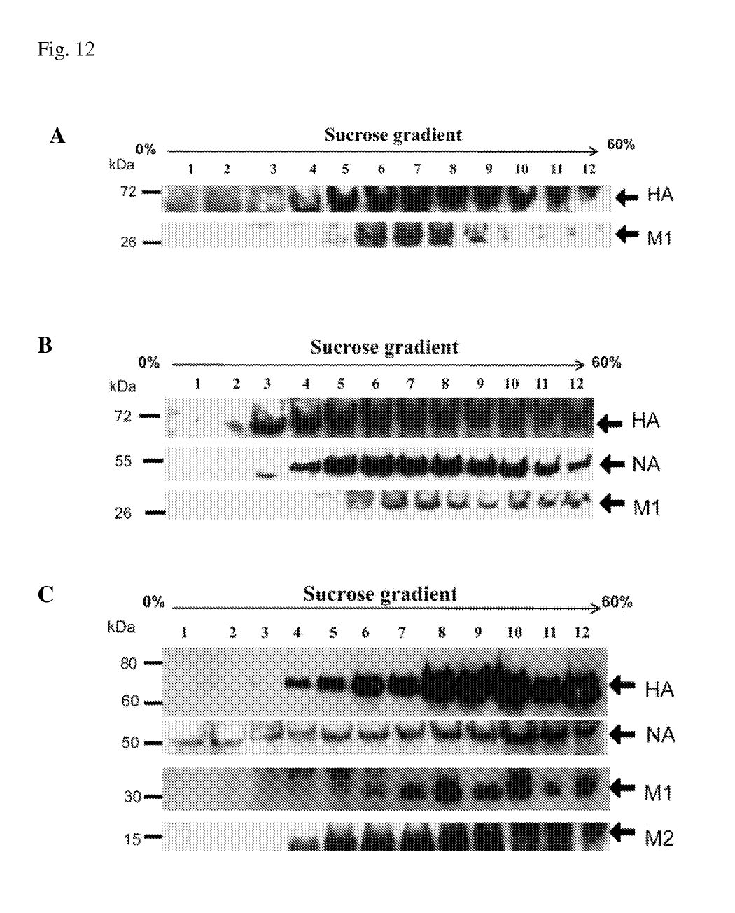

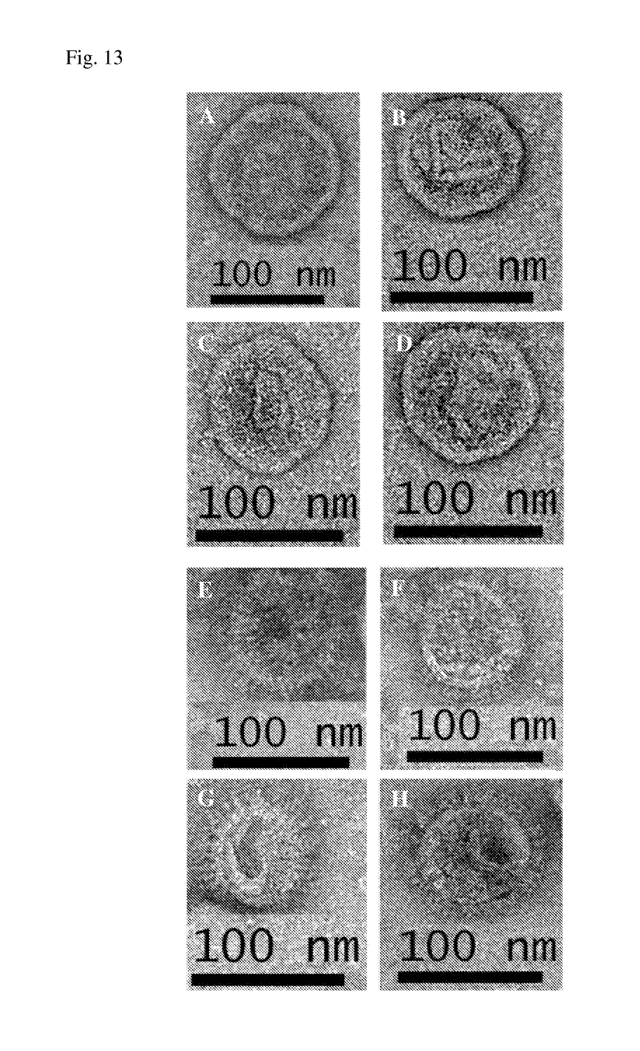

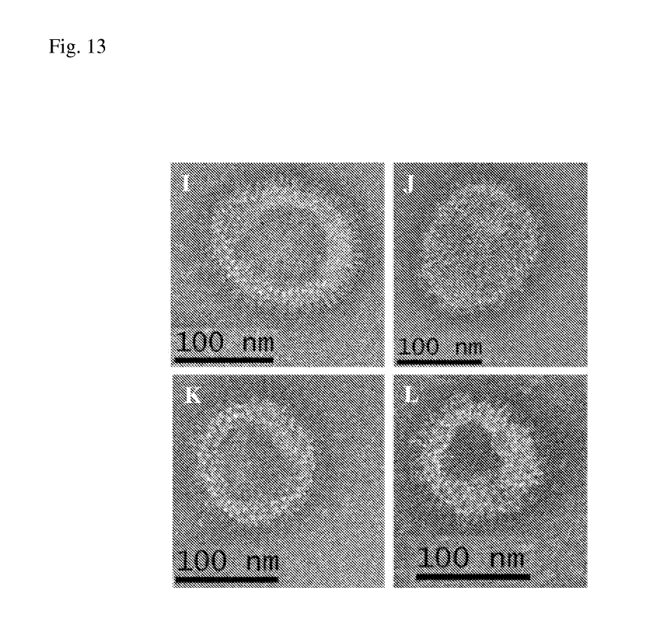

A baculovirus-insect cell expression system was used to prepare the influenza VLPs by the over-expression of two viral proteins (HA, M1), three viral proteins (HA, NA, M1), and four viral proteins (HA, NA, M1, M2). The cDNAs of the four viral proteins were obtained from different influenza virus strains: HA (A/Thailand/1(KAN-1)/2004/H5N1) (SEQ ID NO: 1), NA (A/Viet Nam/1203/2004/H5N1) (SEQ ID NO: 27), M1 (A/WSN/1933/H₁N₁) (SEQ ID NO: 21) and M2 (A/WSN/1933/H₁N₁) (SEQ ID NO: 23). These genes were cloned into the baculovirus vector under two promoters, polyhedron (pH) and p10, to generate a series of recombinant baculoviruses (BacHA-M1, BacNA, BacM2-NA) (FIGS. 11A-C). Influenza VLPs were obtained from Sf9 cells that were infected with BacHA-M1 (two viral proteins), co-infected with BacHA-M1 and BacNA, or co-infected with BacHA-M1 and BacM2-NA. Influenza VLPs were obtained from the culture supernatants and purified by ultracentrifugation and sucrose gradient sedimentation. The formation of influenza VLPs was in the sucrose gradient fractions verified by Western blotting in the presence of two viral proteins HA and M1 (FIG. 12A), three viral proteins HA, NA, M1 (FIG. 12B), and four viral proteins HA, NA, M1, M2 (FIG. 12C). The TEM results reveal that the VLPs obtained from infected Sf9 cells were roughly spherical and were pleomorphic. The average diameters of the influenza VLPs were 102±3 nm (N=10) for two viral proteins, 100±4 nm (N=10) for three viral proteins, and 97±13 nm (N=10) for four viral proteins (FIG. 13). Distinctive influenza spike projections were observed on the surface of the VLPs expressed using three and four viral proteins (FIG. 13). The influenza VLPs that were expressed using two, three and four viral proteins were all capable of maintaining red blood cell agglutination as determined from the HA titers of 512 (two viral proteins), 256 (three viral proteins), and 512 (four viral proteins) per 50 μL.

Production of Influenza VLPs with EGFP/M2 Fusion Protein

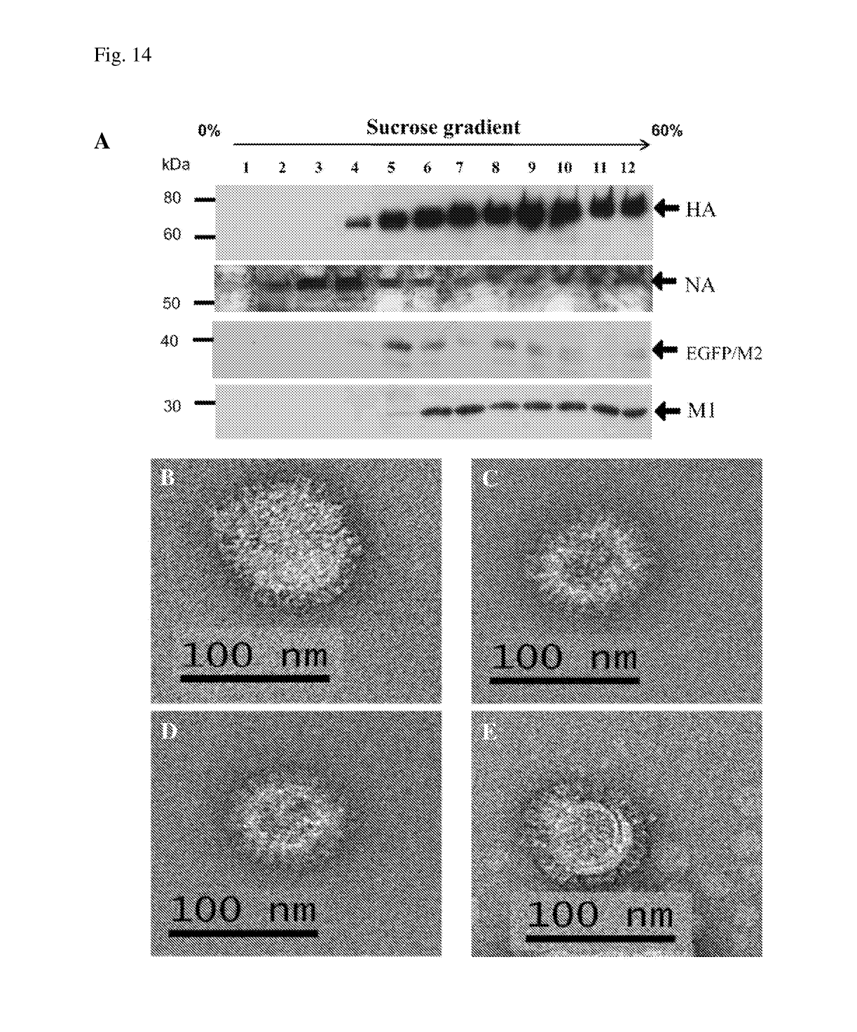

It was proposed that M2 protein can be used as a molecular fabricator (i) without disrupting the assembly of VLPs and (ii) while retaining the native structures of HA and NA envelope proteins on the particle surfaces. Fabrication of influenza VLPs was obtained by the over-expression of four viral proteins by a direct fusion of M2 to EGFP. The EGFP gene was added to the N terminus of the M2 gene to construct the baculovirus (BacEGFP/M2-NA). Sf9 cells were co-infected with two recombinant baculoviruses (BacHA-M1 and BacEGFP/M2-NA) to generate the EGFP-VLPs. Direct fusion of EGFP to M2 did not influence the formation of VLPs as revealed by the presence of four viral proteins in the sucrose gradient fractions (FIG. 14A) and the TEM visualization of the spherical and pleomorphic particles with an average diameter of 93±13 nm (N=10) (FIGS. 14B-E).

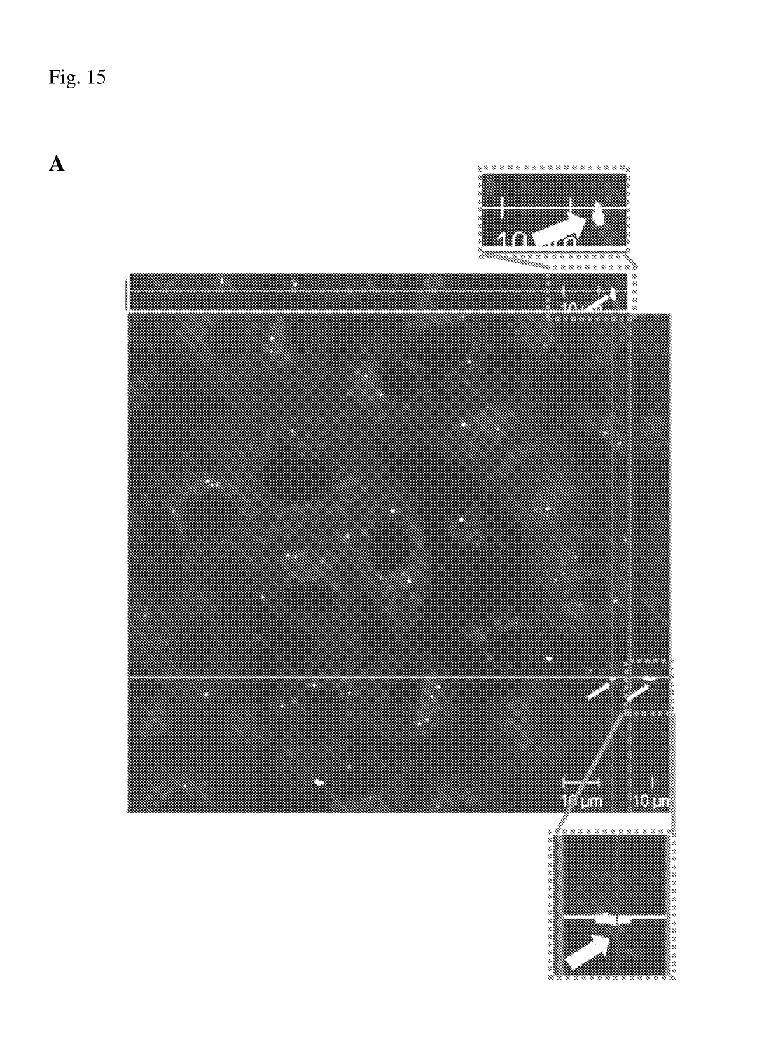

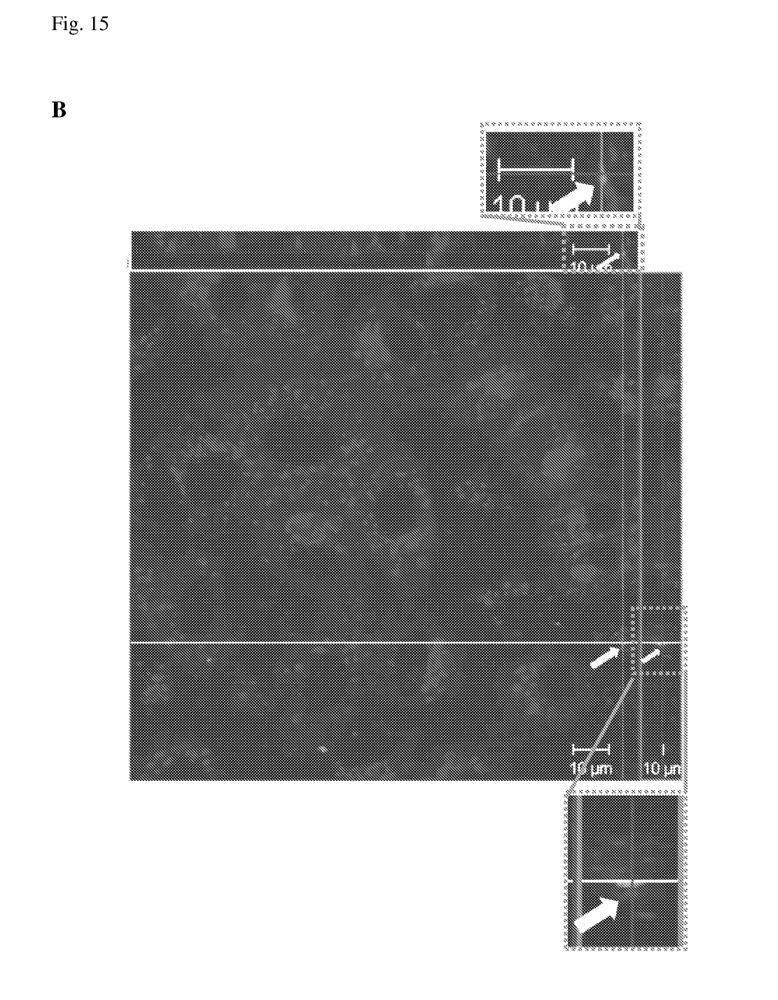

To further show the functionality of the EGFP-VLPs, live cell imaging was used to visualize the uptake of EGFP-VLPs in A549 cells. Using confocal microscopy at various wavelengths of emitted light, green fluorescent spots of the EGFP-VLPs were observed inside the A549 cells with light that was excited at 488 nm (FIG. 15A), and overlapped the red fluorescent spots of the VLPs that were stained with DiI, which is a fluorescent lipophilic dye that was used to label viral membranes within the A549 cells with an excited light wavelength of 561 nm (FIG. 15B). In parallel, A549 cells were labeled with DiD, a fluororescent lipophilic dye for labeling cell membranes, yielding blue fluorescent spots with an excited light wavelength at 633 nm. These results reveal that influenza VLPs can be generated by the M2 fusion of EGFP for imaging single virus entering A549 cells.

Production of Influenza VLPs with Flagellin/M2 and Profilin/M2 Fusion Proteins

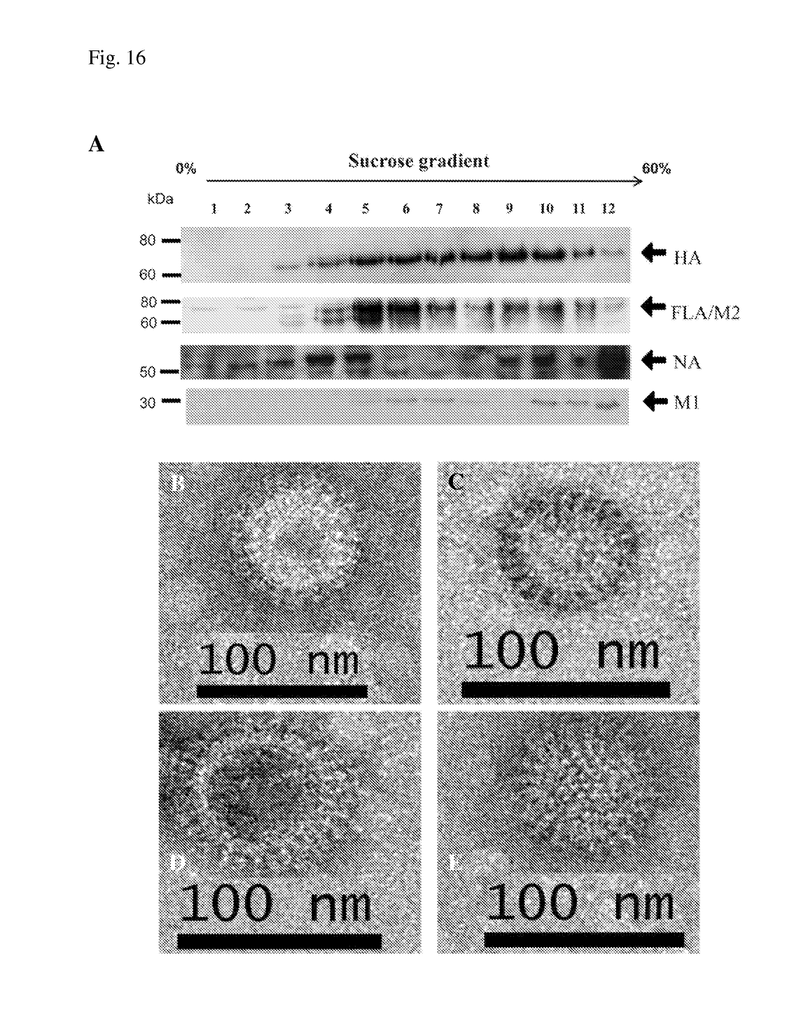

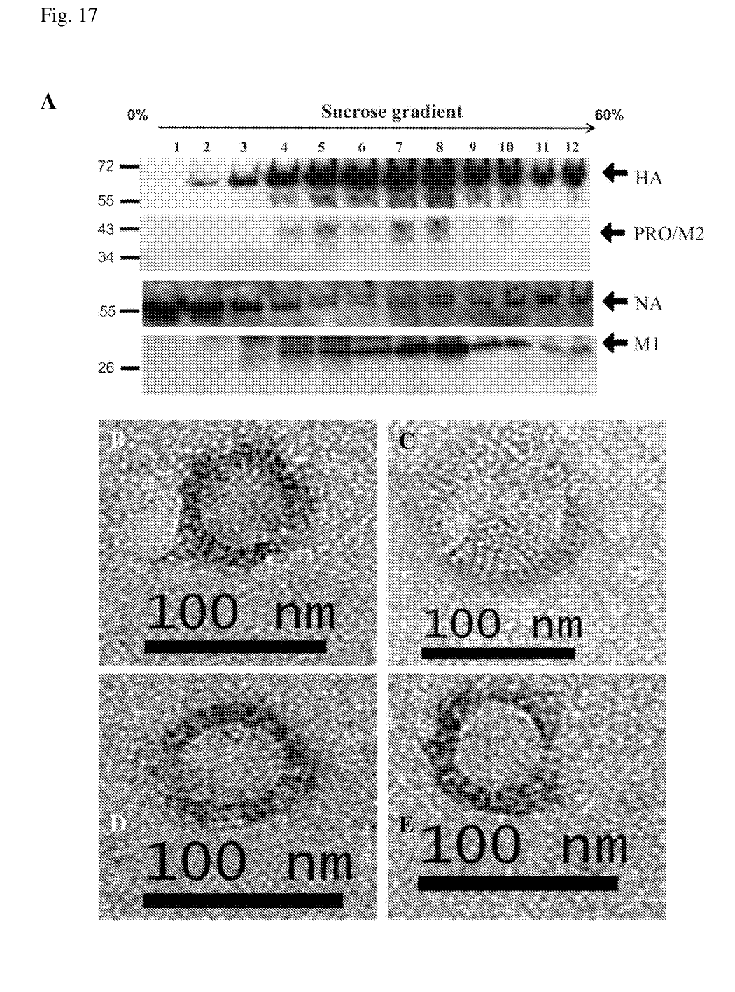

Two molecular adjuvants, FliC and PRO, were then replaced with EGFP to generate two molecular adjuvanted VLPs, FliC-VLPs and PRO-VLPs. The full-length genes of FliC and PRO were fused in front of the M2 gene to construct the recombinant baculoviruses, BacFliC/M2-NA and BacPRO/M2-NA. Sf9 cells were co-infected with BacHA-M1 and Bac FliC/M2-NA or BacHA-M1 and BacPRO/M2-NA to yield FliC-VLPs and PRO-VLPs. Direct fusion of FliC and PROto M2 formed FliC-VLPs (FIG. 16A) and PRO-VLPs (FIG. 17A) as evidenced by the presence of the fusion proteins and other three viral proteins HA, NA, M1 in the sucrose fractionated samples. The morphologies of FliC-VLPs and PRO-VLPs were spherical and pleomorphic, with average diameters of 94±7 nm (N=10) and 94±13 nm (N=10), respectively (FIGS. 6B-E and 17B-E). These results reveal that the molecular adjuvanted VLPs can be obtained using M2 fusion proteins.

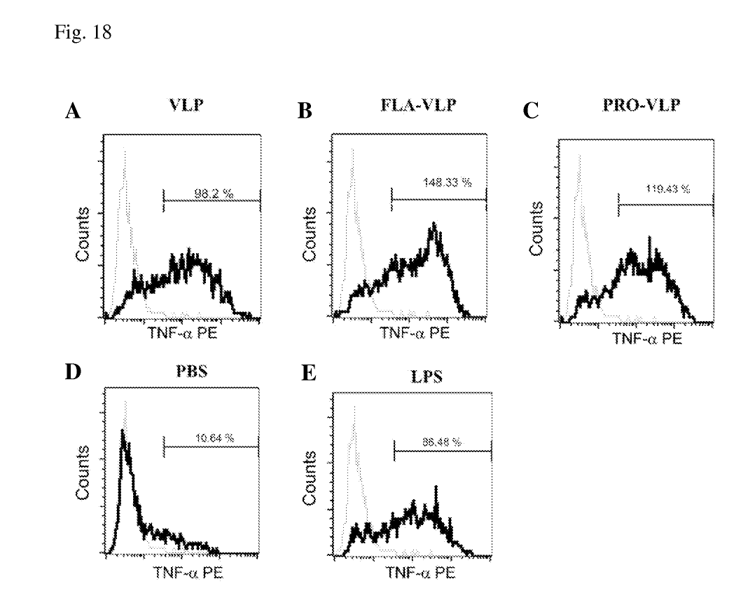

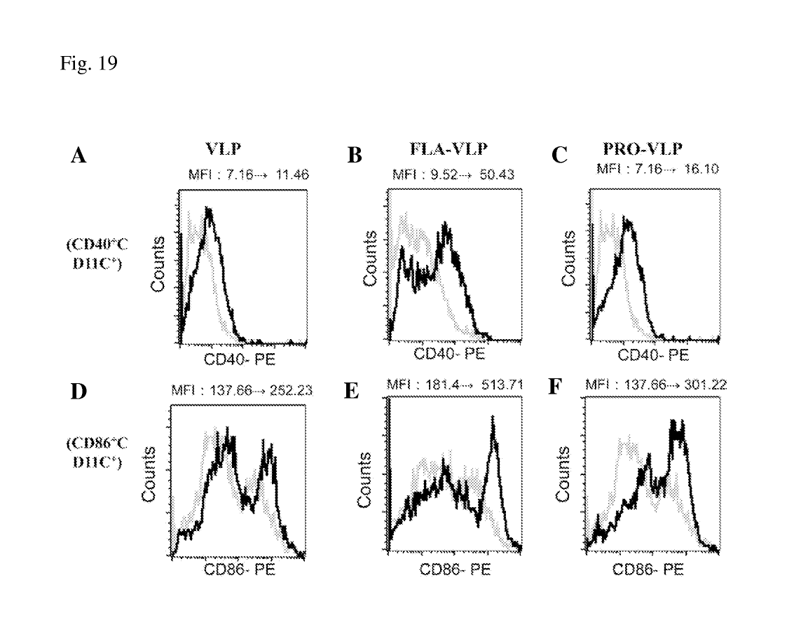

To study the effects of molecular adjuvanted VLPs on dendritic cells, mouse BMDCs were obtained in vitro, treated with various influenza VLPs (VLPs, FliC-VLPs, PRO-VLPs) and then analyzed using FACS analysis. The results indicate that the production of TNF-α in BMDCs increased from 98.2% (VLP) to 148.3% (FliC-VLP) and 119.4% (PRO-VLP) than in the controls of untreated (10.6%) and LPS-treated BMDC cells (86.5%) (FIG. 18). The maturation of BMDCs that was caused by influenza VLPs was also elucidated by measuring the amount of the co-stimulatory molecules of CD40 and CD86 on the surfaces of BMDCs. The results show that since the mean fluorescence intensities (MFI) of CD40⁺CD11c⁺ and CD86⁺CD11c⁺in BMDCs upon treatment with FliC-VLPs and PRO-VLPs increased above those in VLPs (FIG. 19), the molecular adjuvanted VLPs (FliC-VLPs and PRO-VLPs) induced BMDCs to produce more TNF-α and to promote more DC maturation in vitro.

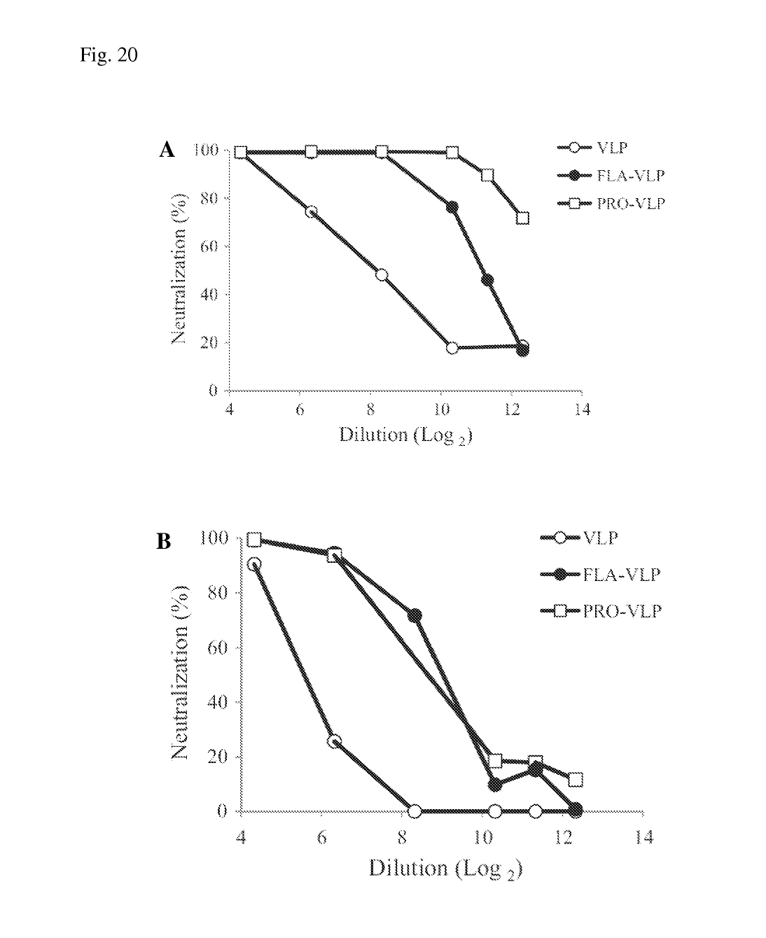

To investigate whether immunization with the molecular adjuvated FliC-VLPs and PRO-VLPs can elicit more potent immune responses than the wild-type VLPs, BALB/c mice were immunized with VLPs, FliC-VLPs, and PRO-VLPs at 15 μg (total protein) per dose for three immunizations. The mouse sera were collected one week after the third immunization and analyzed for HSpp neutralization. The results show that the antisera that were collected from mice that have been immunized by VLPs, FliC-VLPs and PRO-VLPs neutralized HSpp of the homologous KAN-1 strain (FIG. 20A) and the heterologous Anhui strain (FIG. 20B) were all in a dose-dependent manner. For neutralization of the homologous strain, the 50% neutralization titers were log₂ 6.5 for VLP antisera, log₂ 11.2 for FliC-VLP antisera, and log₂ 12.8 for PRO-VLP antisera. For neutralization of the heterologous Anhui strain, the 50% neutralization titers were log₂ 5.7 for VLP antisera, log₂ 8.8 for FliC-VLP antisera, and log₂ 9.3 for PRO-VLP antisera. Immunization using the fabricated VLPs that contained the molecular adjuvants (PRO-VLPs and FliC-VLPs) elicited more potent neutralizing antibody responses in mice against the homologous and the heterologous H5N1 viruses than the wild-type VLPs.

### Example 3

To enhance immunogenicity of vaccines, VLPs comprising GMCSF-fused M2 protein (GMCSF VLPs) or combinational GMCSF/FliC-fused M2 protein (GMCSF/FliC VLPs) were also further selected.

### Material and Methods

### Plasmid Construction

The pFastBac Dual pasmids containing HA/M1, NA/M2, NA/FliC-M2 were constructed as previously reported (Wei H J et al., Vaccine 29 (2011): 7163-7172). In brief, HA gene of A/Thailand/1 (KAN-1)/2004 (H5N1) and M1 gene of A/WSN/1933 (H₁N₁) were cloned into a pFastBac Dual vector (Invitrogen), while NA gene of A/Viet Nam/1203/2004 (H5N1) strain and M2 gene of A/WSN/1933 (H₁N₁) strain were cloned into another one. For molecular adjuvanted VLPs, FliC or GMCSF fused to the N-terminal of the M2 gene was conjugated with NA, and then cloned into the pFastBac Dual vector. All of the manners regarding FliC-VLP are the same as EXAMPLE 2 described. Regarding to the GMCSF-M2 (SEQ ID NO: 30) construction, the cDNA of GMCSF was also fused to the N-terminal of the M2 and further conjugated with NA, and then cloned into the pFastBac vector.

### Production and Purification of Influenza VLPs

All of the manners are the similar as EXAMPLE 2 described. Additionally, to generate dual adjuvanted VLPs, Sf9 cells were co-infected with rBVs expressing HA/M1, FliC-M2/NA or/and GMCSF-M2/NA. At 72 hpi, supernatants containing VLPs (WT VLPs, FliC VLP, GMCSF VLPs or combinational GMCSF/FliC VLPs) were harvested and the purification steps of the VLPs followed the manual.

### Sodium Dodecyl Sulfate Polyacrylamide Gel Electrophoresis (SDS-PAGE) and Western Blotting

All of the manners are the same as EXAMPLE 2 described.

### Mouse Immunization

6 to 8 week-old female BALB/c mice were purchased from National Laboratory Animal Center. Five mice of each group were immunized with 0.05 mg or 0.5 mg HA contents of purified WT VLPs, FliC VLPs, and GMCSF/FliC VLPs combined with 300 ug Alum adjuvant, separately. Immunizations were performed by intramuscular (i.m.) injection at a three weeks interval (weeks 0 and 3). Blood was collected and serum was isolated 14 days after the final booster dose. Sera samples were inactivated by incubation at 56° C. for 30 min and stored at −20° C. for further analyses. All animal experiments were conducted in accordance with guidelines established by the Laboratory Animal Center of National Tsing Hua University (NTHU). Animal use protocols were reviewed and approved by the NTHU Institutional Animal Care and Use Committee (approval no. 10002).

### Enzyme-Linked Immunosorbent (ELISA) Assay

All of the manners are the similar as EXAMPLE 1 described. And the optical density at 450 nm were detected after reacting with anti-mouse IgG, IgG1, IgG2a and IgM antibodies (BioLegend). The reciprocal of highest sera dilution which gave an O.D.450 five-fold higher than that of negative control was designated as antibody end-point titer.

### H5N1 Pseudotyped Particle (H5N1 Pp) Neutralization Assays

HEK293A cells were co-transfected with pNL-Luc-E⁻R⁻, pcDNA3.1(+) expressing the HAs of A/Thailand/1(KAN-1)/2004 (cladel), A/bar-headed goose/Qinghai/1A/2005 (clade 2.2) or A/Anhui/1/2005 (clade 2.3.4) and pcDNA4B expressing the NA of the A/Viet Nam/1203/2004 strain. Culture supernatants were harvested and concentrated following 48 hr transfection, followed by titration of the luciferase activity of H5N1 pp transduction. Further, diluted Sera (two-fold serial dilution, starting from 1:20) were mixed with 50 TCID₅₀ H5 pp for 1 h at 37° C. in 5% CO₂, and then infected the MDCK cell line for 48 h. After 48 h.p.i., cells were lysed with glo lysis buffer (Promega) and luciferase activity was measured via neolite luciferase substrate (PerkinElmer). Neutralizing antibody titers were quantified as reduced luciferase expression level following H5 pp transduction in MDCK cells, and logIC50 was determined by the diluted serum concentration to obtain a 50% reduction in RLU compared to control wells containing the virus only.

### Hemagglutinin Inhibition (HI) Assays

Sera were treated with receptor-destroying enzyme (Denka Seiken) for 18 h at 37° C. followed by 56° C. for 30 min to inactivate enzyme activity. Treated sera were two-fold serially diluted (starting from 1:10) and incubated with 4 HA units of H5N1 pp containing HA from the KAN-1 and Qinghai strains. Next, 0.5% turkey red blood cells were added and incubated for another 30 min at room temperature. HI titer was measured as the reciprocal of the highest dilution of sera which completely inhibited hemagglutination.

### Detection of HA-Specific Antibodies Secreting B-Cells

Multiscreen 96-well filtration plates were coated with 1 μg of recombinant HA proteins and incubated at 4° C. overnight. Plates were blocked with complete RPMI-1640 (10% FBS, 1×P/S, 1× Sodium pyruvate, 1×NEAA and 0.1% β-ME) for 2 hr at RT. 5×10⁵ splenocytes per well were added into 96 well plates at 37° C. with 5% CO₂ humidified incubator for 48 hrs. After 3 times washes by PBST, HRP-conjugated anti-mouse IgG antibodies were added to plates and incubated at RT for 2 hours. After another 3 times washes by PBST and 2 times by PBS, AEC substrate was added to the plates and developed at RT for 1060 minutes and stopped the reaction by ddH₂O.

### Enzyme-Linked Immunospot (ELISPOT) Assay

T cell responses were measured by mouse IFN-γ and IL-4 ELISPOT kit (eBioscience). In brief, multiscreen 96-well filtration plates were coated with anti-mouse IFN-γ or IL-4 capture antibodies and incubated at 4° C. overnight. Block plates with complete RPMI-1640 for 1 hour at RT. 5×10⁶ celUml were seeded and stimulated with 2 μg recombinant H5HA proteins for 48 hours. After 3 times washes by PBST, detection antibody was added to plates and incubated at RT for another 2 hrs. Wash 3 times and Avidin-HRP was added into plates at RT for 45 minutes. After 3 times washes by PBST and 2 times by PBS, AEC substrate was added to the plates and developed at RT for 1060 minutes and stopped the by ddH₂O.

### Statistic Analysis

All results were analyzed using Student's t tests with a P value indicating a statistical significance. Asterisk (*) in the Fig.s represents a statistically significant difference. * means p<0.05; ** indicates p<0.01.

### Results

Production of Influenza VLPs with GM-CSF/M2 and Flagellin/M2 Fusion Proteins

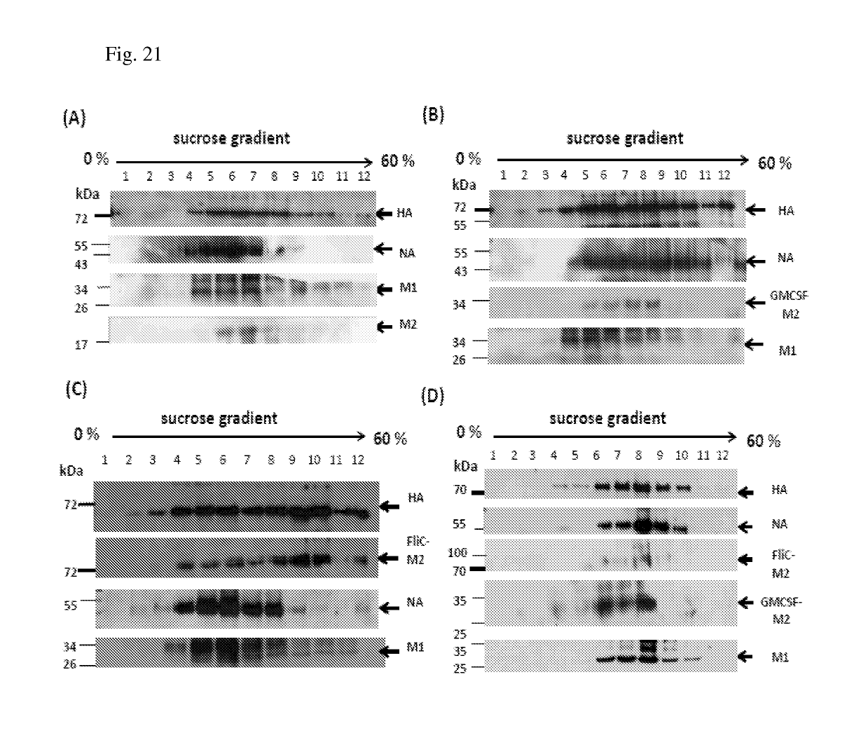

Influenza VLPs were obtained from Sf9 cells co-infected with recombinant baculoviruses (Bac-HA-M2 and Bac-NA-M1) and the culture supernatant was further purified by 0-60% sucrose gradient sedimentation. Purified influenza VLPs in sucrose gradient fractions were verified by Western blotting, showing that the fractions 5 to 8 contained four viral structure proteins: HA, NA, M1, and M2 (FIG. 21(A)). Additionally, the gene of GMCSF or FliC was fused to the N-terminal end of the M2 coding sequence to construct baculoviruses of Bac-HA-GMCSF/M2 and Bac-HA-FliC/M2. Influenza VLPs containing each M2 fusion protein was obtained from baculovirus-infected Sf9 cells co-infected with Bac-HA-GMCSF/M2 and Bac-NA-M1 for GM-CSF VLPs, co-infected with Bac-NA-FliC/M2 and Bac-NA-M1 for FliC VLPs, and co-infected with Bac-HA-GM-CSF/M2, Bac-NA-FliC/M2 and Bac-NA-M1 for GM-CSF/FliC VLPs. The M2-fused GM-CSF VLPs, FliC VLPs and GMCSF/FliC VLPs were shown in Western blots, showing the presence of M2 fusion proteins and other three viral proteins of HA, NA, and M1 in sucrose fractions (FIG. 21(B), (C), (D)). These results indicate that influenza VLPs containing M2 fusion proteins of GM-CSF, FliC, GM-CSF/FliC can be obtained without affecting VLP assembly and production.

### HA-Specific Antibody Responses Elicited by H5N1 VLP Immunizations

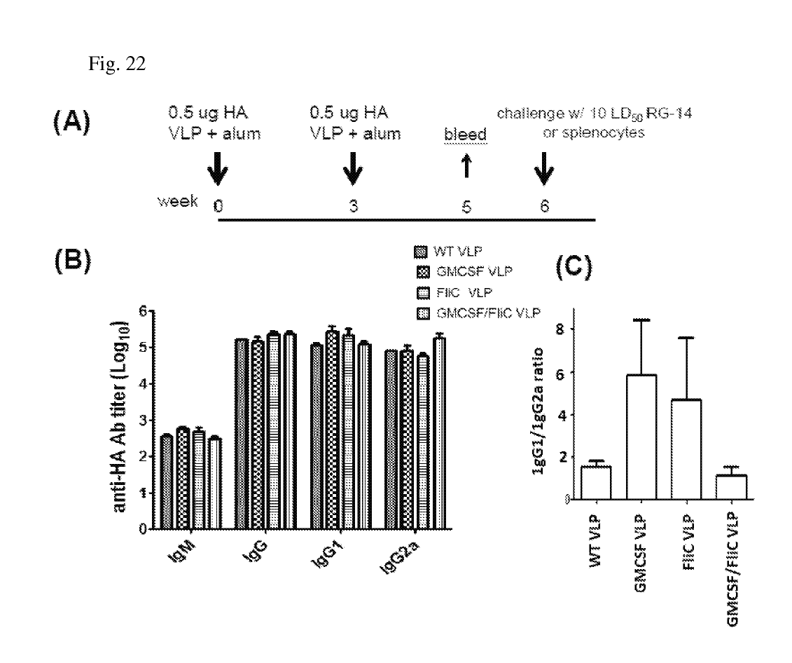

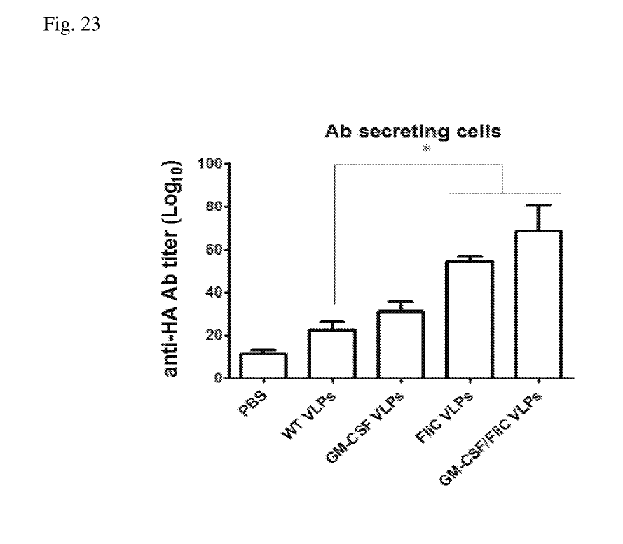

Groups of 6 to 8 weeks old female BALB/c mice were intramuscularly immunized with the influenza VLPs, GM-CSF VLPs, FliC VLPs, and GM-CSF/FliC VLPs at 0.5 g HA content plus 300 g Alum for two doses at a three-week interval (FIG. 22(A)). Sera were collected two weeks after the second dose immunization. The results showed that the HA-specific IgM and IgG titers elicited by these immunizations had no significant differences, neither did the subtypes of IgG1 and IgG2a (FIG. 22(B)). However, the ratios of IgG1 to IgG2a for GM-CSF VLP and FliC VLP groups were significantly higher than those of GM-CSF/FliC VLPs and WT VLP groups (FIG. 22(C)), suggesting Th2 skewed responses by GM-CSF VLP and FliC VLP immunizations. We further measured HA-specific antibody-secreting B-cells (ASCs) from splenocytes collected 3 weeks after the second dose immunization, following stimulation by 1 μg pooled HA peptides and the quantities of IgG-secreting cells were determined using ELISPOT assay. HA-specific ASCs increased following the immunizations using GMCSF/FliC VLPs and FliC VLPs (FIG. 23)). These results suggest the use of FliC fused on M2 of VLP may elicit a more potent B cell response in mice.

### Hemagglutination Inhibition (HI) and Neutralizing Antibody Titers Against the Homologous and Heterologous Clades of H5N1 Viruses

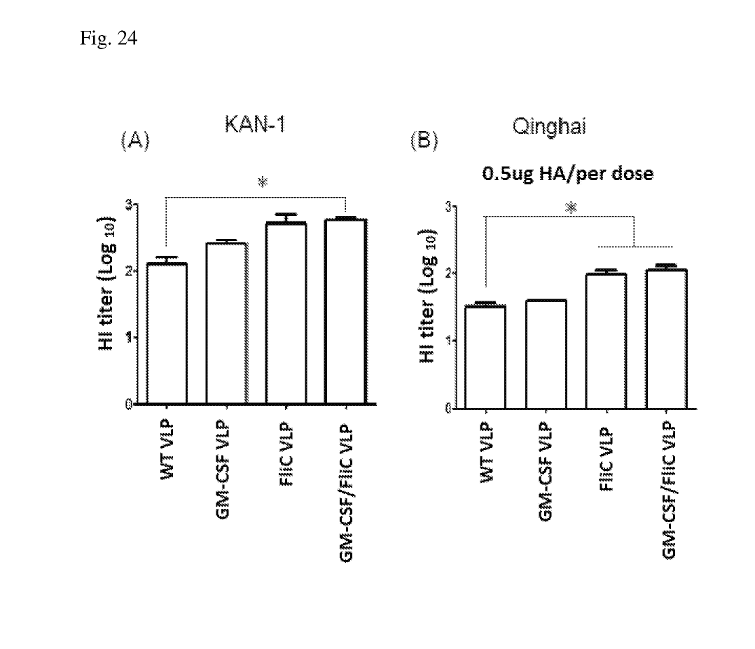

Lentivirus pseudotype H5N1 particles were used to determine the titers of HI and neutralizing antibodies. Sera were two-fold serially diluted (starting from 1:10) and incubated with 4 HA units of H5N1 pp containing HA from H5N1 KAN-1 (clade 1), Qinghai (clade 2.2) and Anhui (clade 2.3.4) strains. The results indicate that GMCSF/FliC VLP immunization elicited a significantly higher HI titer against the homologous KAN-1 strain as compared to WT VLP immunization (FIG. 24(A)). FliC VLP and GMCSF/FliC VLP immunizations also elicited higher HI titers against the heterologous Qinghai strain (FIG. 24(B)). No significant HI titers were observed against the heterologous Anhui strain (data not shown).

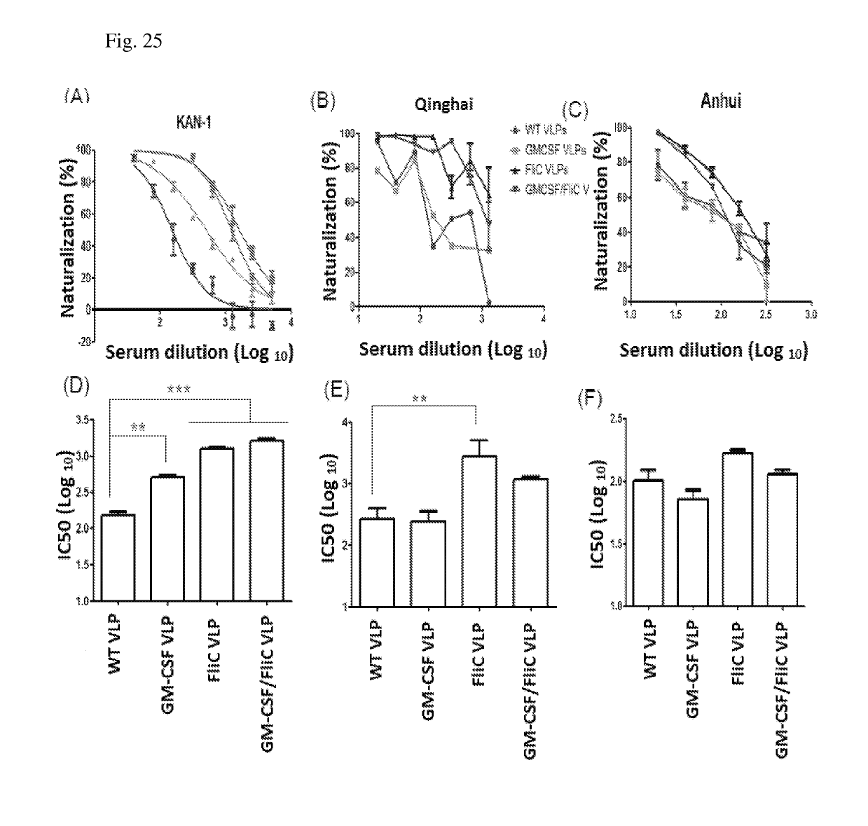

A luciferase-based H5N1 pp assay was further used to measure the neutralizing antibody titers against the homologous (KAN-1, clade 1) and heterologous (Qinghai, clade 2.2; Anhui, clade 2.3.4) strain. Serum dilution-dependent neutralization curves were obtained for all groups of immunization by WT VLPs, GMCSF VLPs, FliC VLPs or GMCSF/FliC VLPs against KAN-1 H5N1 pp (FIG. 25(A)), Qinghai H5N1 pp (FIG. 25(B)) and Anhui H5N1 pp (FIG. 25(C)). The corresponding IC50 values obtained from these neutralization curves against the homologous H5N1 KAN-1 virus followed the orders of GM-CSF/FliC VLP>FliC VLP>GM-CSF VLP>WT VLP (FIG. 25(D)). The IC50 titers against two heterologous H5N1 virus strains also followed the orders of FliC VLP>GM-CSF/FliC VLP>GM-CSF VLP and WT VLP against Qinghai, clade 2.2 (FIG. 25E) and Anhui, clade 2.3.4 (FIG. 25(F)). Therefore, FliC VLPs and GM-CSF/FliC VLPs induced more potent HI and neutralizing antibodies against the homologous and heterologous strains of H5N1 viruses.

### T-Cell Responses Elicited by H5N1 VLP Immunizations

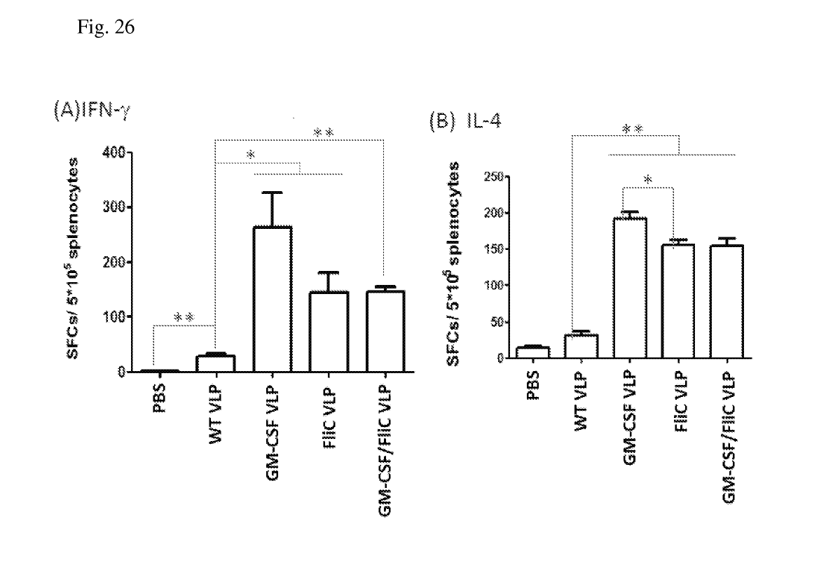

To measure T cell responses induced by immunizations of WT VLPs, GMCSF VLPs, FliC VLPs or GMCSF/FliC VLPs, splenocytes obtained from each group of immunized mice were stimulated with a mixture of H5HA peptides and the quantities of IFN-γ and IL-4-secreting T cells were determined using ELISPOT assay. Our results indicated that the immunization groups using GMCSF VLPs, FliC VLPs, and GMCSF/FliC VLPs produced significantly higher numbers of IFN-γ-secreting T cells as compared to the WT VLPs group (FIG. 26(A)). The immunization groups using GMCSF VLPs, FliC VLPs, and GMCSF/FliC VLPs also had higher numbers of IL4-γ-secreting T cells as compared to WT VLPs group (FIG. 26(B)). Noticeably, the numbers of IL4-γ-secreting T cells by GM-CSF VLPs immunization were statistically higher than that by FliC VLPs immunization (FIG. 26(B)). Therefore, GMCSF VLPs induced the strongest T cell responses (Th1 and Th2) among all four immunization groups, suggesting GM-CSF VLPs triggered more potent T-cell activation.

What is claimed is:

1. An influenza virus-like particle, which is used to enhance immunogenicity of vaccines, comprising an adjuvant-fused M2 protein.

2. The influenza virus-like particle of claim 1, which elicits an immune response against a plurality of avian influenza virus subtypes in a subject.

3. The influenza virus-like particle of claim 1, which is used as a booster to a DNA vaccine.

4. The influenza virus-like particle of claim 1, which further comprises M1 protein, HA protein and NA protein.

5. The influenza virus-like particle of claim 1, wherein the adjuvant is flagellin (FliC), profilin (PRO), Granulocyte macrophage colony-stimulating factor (GM-CSF), or a combination thereof.

6. The influenza virus-like particle of claim 5, wherein the adjuvant is a combination of GM-CSF and FliC.

7. The influenza virus-like particle of claim 1, which is derived from cell infected by recombinant baculoviruses comprising one or more plasmids containing a gene encoding an adjuvant-fused M2 protein, wherein the adjuvant is flagellin (FliC), profilin (PRO), Granulocyte macrophage colony-stimulating factor (GM-CSF), or a combination thereof.
